SEQUENCE LISTING (4 sequences)

SEQ ID NO: 1 - DNA, 23 bp, Artificial Sequence
  Bone sialoprotein forward primer
aagcaatcaccaaaatgaagact

SEQ ID NO: 2 - DNA, 24 bp, Artificial Sequence
  Bone sialoprotein reverse primer
tggaaatcgttttaaatgaggata

SEQ ID NO: 3 - DNA, 22 bp, Artificial Sequence
  GAPDH forward primer
gggcatcctgggctacactgag

SEQ ID NO: 4 - DNA, 19 bp, Artificial Sequence
  GAPDH reverse primer
ggcccctcccctcttcaag

(19) United States
(12) Patent Application Publication
(10) Pub. No.: US 2005/0227219 A1
(43) Pub. Date: Oct. 13, 2005

(54) Method for suppression or reversing of cellular aging

(75) Inventors: Vladimir Volloch, Brookline, MA (US); David Kaplan, Concord, MA (US)

Correspondence Address: PALMER & DODGE, LLP;KATHLEEN M. WILLIAMS, 111 HUNTINGTON AVENUE, BOSTON, MA, 02199, US

(73) Assignee: Tufts University

(21) Appl. No.: 11/022,452

(22) Filed: Dec. 22, 2004

Related U.S. Application Data
(63) Continuation of application No. PCT/US03/19577, filed on Jun. 20, 2003.
(60) Provisional application No. 60/391,768, filed on Jun. 26, 2002.

(51) Int. Cl. A01N 1/02; C12N 5/08

(52) U.S. Cl. 435/2; 435/366; 435/404

(57) ABSTRACT

A method is provided for growing young cells that reduces and/or reverses age-related processes that would otherwise occur in those cells, and also compositions for growing cells in such a method.

### BACKGROUND

Biomaterial surface morphology and chemistry influence cell responses mediated via signaling cascades that regulate a wide range of metabolic processes. These responses range from changes in surface adhesion and cell spreading through membrane integrins receptors, and reconstruction or remodeling of the extracellular matrix through catabolism and biosynthesis of new scaffolding to activation of cytokine, cytoskeletal and other biochemical pathways regulating or modulating cellular morphology and function. To date, the elucidation of the relationships between biomaterial surfaces and cell responses has focused primarily on changes in cell adhesion and spreading, on apoptosis responses, or on specific cell functions such as mineralization. Thus, Chen and co-workers reported that surface geometry had a direct impact on capillary endothelial cell survival measured by the apoptosis response (Chen et al., 1997). In other studies, the adhesion, spreading and mineralization of osteoblasts on quartz surfaces were influenced by the density of the cell binding domain RGD coupled to the surface (Rezania and Healy, 2000), and surface microtopography with poly(glycolic-co-lactic) acid modified with collagen was shown to influence the adhesion and migration of HepG2 cells (Ranucci and Moghe, 2001).

Cellular aging and the stress response potential appear to be intimately related. In human and animal cells, aging is associated with the loss of the potential to respond to stresses. In young cells, transient exposure of cells or organisms to a mild stress confers resistance against subsequent exposure to a severe stress of the same type or of different types, a phenomenon known as acquired stress tolerance. On the molecular level, it has been shown that the acquired tolerance is accounted for by accumulation of the major stress response protein, Hsp70 (heat shock protein of 70 kDa) and other Hsps (Volloch et al., 1998). Under normal physiological conditions, stress is usually elevated gradually, and cells develop acquired tolerance while stress is still mild; it protects cells at later severe stages of a stress. Thus, stress-inducible Hsp70 expression, which is responsible for acquired stress tolerance, represents one of the major cellular protective systems. However, this line of defense is being progressively weakened and lost with aging. The construction of a tissue, including sometimes massive in vitro expansions of a relatively few stem cells, may involve a substantial number of cell divisions, resulting in cells that are “old” by the time of implantation. The aging of cells during tissue engineering represents a significant problem that may compromise the usefulness of engineered tissue because of the aging-dependent attenuation of some cellular functions, such as the ability to respond to stresses (Volloch et al., 1998), and of the potential to undergo differentiation.

A large number of recent studies indicated an intimate, moreover, probably a causal, relationship between the potential to respond to stresses and cellular aging with the former strongly influencing the Tate of the latter. Such a notion is supported by two lines of observations. First, in human and animal cells stress response is attenuated in an age-dependent manner. “Cellular age” is often expressed by the number of cell doublings, and a typical human cell can undergo approximately 70 divisions. The second line of evidence for the notion that cellular aging and the potential of stress response are causally related is constituted by studies employing genetic manipulations on lower organisms, where it has been convincingly demonstrated that genetic manipulations can lead to significant life extension. Practically all life span extending mutations confer stress-resistant phenotype, while the reverse is also true, namely the selection for stress resistance results in the alleles conferring extended life span (Walker et al., 1998). The addition of OS to BMSC cultures resulted in significant increase in the activity of an “early” osteogenic marker, alkaline phosphatase, in young cells, reflecting the degree of progression into the osteoblastic lineage (Jaiswal et al., 1997). With mesenchimal stem cells, it has been shown that osteogenic potential declines with prolonged cultivation, i.e., cellular aging (Bruder et al., 1997). It can be argued that if the rate of cellular aging is reduced, the potential to differentiate will be retained to a higher extent. It has also been recently demonstrated that in aged cells one of the key aging-related processes previously considered irreversible, attenuation of the expression of a major stress response protein, Hsp70, can be reversed.

Manipulation of the potential for stress response may interfere with the process of cellular aging in human and animal cells. Recently, it was reported that growth of cells on a collagen matrix markedly enhanced the resistance of cells to stresses (Howell and Doane, 1998; Hoyt et al., 1995; Aoshiba et al., 1997; Cao et al., 1999; Mooney et al., 1999). Reports also indicate that native, non-denatured collagen may have a deleterious effect on cells by suppressing their proliferation (Henriet et al., 2000). A number of factors, among them type of tissue culture plastic, are known to affect the developmental potential of cultured cells (Maniatopolous et al., 1988; Aronow et al., 1990; LeBoy et al., 1991; Haynesworth et al., 1992a, 1992b; Gallagher et al., 1996). Another factor that can affect differentiation potential of cells is a high cell density (Caplan et al., 1983).

### SUMMARY OF THE INVENTION

The present invention relates to a method for growing young cells that reduces and/or reverses age-related processes that would otherwise occur in those cells. As disclosed herein, growth of cells on a substrate lacking in its normal higher-order structure, e.g., a substrate that has been denatured or disorganized, e.g., a disorganized or denatured polymer matrix, results in a reversal of age-related processes, and/or maintenance of non-age related processes in cells.

The invention features a method of preserving one or more cellular functions that are characteristic of cells in a non-senescent state, where the one or more cellular functions are lost in cells that are in a senescent state, where the method comprises (a) providing cells that possess one or more cellular functions that are characteristic of cells in a non-senescent state; (b) providing a matrix of denatured polymer (e.g., denatured type I collagen, denatured type I collagen of between 0.1 mg/ml and 5 mg/ml, denatured type I collagen of 0.5 mg/ml, or denatured type I collagen of 0.3 mg/ml); and (c) culturing the cells of (a) on the matrix of (b) under conditions sufficient to preserve the one or more cellular functions that are characteristic of cells in a non-senescent state; thereby preserving the one or more cellular functions that are characteristic of cells in a non-senescent state. The cells can be stem cells, primary cells, or bone marrow cells. The cellular function can be cell plasticity, differentiation potential, β-galactosidase expression, alkaline phosphatase expression, bone sialoprotein expression, calcium deposition, or heat shock protein expression.

In addition, the invention features a method of restoring one or more cellular functions that are characteristic of cells in a non-senescent state, where the one or more cellular functions are lost in cells that are in a senescent state, and where the method includes: (a) providing cells that have lost one or more cellular functions that are characteristic of cells in a non-senescent state; (b) providing a matrix of denatured polymer (e.g., denatured type I collagen, denatured type I collagen of between 0.1 mg/ml and 5 mg/ml, denatured type I collagen of 0.5 mg/ml, or denatured type I collagen of 0.3 mg/ml); and (c) culturing the cells of (a) on the matrix of (b) under conditions sufficient to restore the one or more cellular functions that are characteristic of cells in a non-senescent state; thereby restoring the one or more cellular functions that are characteristic of cells in a non-senescent state. The cells can be stem cells, primary cells, or bone marrow cells. The cellular function can be cell plasticity, differentiation potential, β-galactosidase expression, alkaline phosphatase expression, bone sialoprotein expression, calcium deposition, or heat shock protein expression.

The invention also features a method of preserving cells in a non-senescent state, where the method comprises (a) providing cells in a non-senescent state; (b) providing a matrix of denatured polymer (e.g., denatured type I collagen, denatured type I collagen of between 0.1 mg/ml and 5 mg/ml, denatured type I collagen of 0.5 mg/ml, or denatured type I collagen of 0.3 mg/ml); and (c) culturing the cells of (a) on the matrix of (b) under conditions sufficient to preserve the cells in a non-senescent state; thereby preserving the cells in a non-senescent state. The cells can be stem cells, primary cells, or bone marrow cells.

In another aspect, the invention features a method of restoring cells to a non-senescent state, where the method includes: (a) providing cells in a senescent state; (b) providing a matrix of denatured polymer (e.g., denatured type I collagen, denatured type I collagen of between 0.1 mg/ml and 5 mg/ml, denatured type I collagen of 0.5 mg/ml, or denatured type I collagen of 0.3 mg/ml); and (c) culturing the cells of (a) on the matrix of (b) under conditions sufficient to restore the cells to a non-senescent state; thereby restoring the cells to a non-senescent state. The cells can be stem cells, primary cells, or bone marrow cells.

In another aspect, the invention features a method of preserving the plasticity of cells, where the method comprises (a) providing cells that possess plasticity; (b) providing a matrix of denatured polymer (e.g., denatured type I collagen, denatured type I collagen of between 0.1 mg/ml and 5 mg/ml, denatured type I collagen of 0.5 mg/ml, or denatured type I collagen of 0.3 mg/ml); and (c) culturing the cells of (a) on the matrix of (b) under conditions sufficient to preserve the plasticity of the cells; thereby preserving the plasticity of the cells. The cells can be stem cells, primary cells, or bone marrow cells.

In a further aspect, the invention features a method of restoring the plasticity of cells, where the method comprises: (a) providing cells that have lost plasticity; (b) providing a matrix of denatured polymer (e.g., denatured type I collagen, denatured type I collagen of between 0.1 mg/ml and 5 mg/ml, denatured type I collagen of 0.5 mg/ml, or denatured type I collagen of 0.3 mg/ml); and (c) culturing the cells of (a) on the matrix of (b) under conditions sufficient to restore the plasticity of the cells; thereby restoring the plasticity of the cells. The cells can be stem cells, primary cells, or bone marrow cells.

In a further aspect, the invention features a method of preserving the differentiation potential of cells, where the method comprises (a) providing cells that possess differentiation potential; (b) providing a matrix of denatured polymer (e.g., denatured type I collagen, denatured type I collagen of between 0.1 mg/ml and 5 mg/ml, denatured type I collagen of 0.5 mg/ml, or denatured type I collagen of 0.3 mg/ml); and (c) culturing the cells of (a) on the matrix of (b) under conditions sufficient to preserve the differentiation potential of the cells; thereby preserving the differentiation potential of the cells. The cells can be stem cells, primary cells, or bone marrow cells.

In another aspect, the invention features a method of restoring the differentiation potential of cells, the method comprising: (a) providing cells that have lost differentiation potential; (b) providing a matrix of denatured polymer (e.g., denatured type I collagen, denatured type I collagen of between 0.1 mg/ml and 5 mg/ml, denatured type I collagen of 0.5 mg/ml, or denatured type I collagen of 0.3 mg/ml); and (c) culturing the cells of (a) on the matrix of (b) under conditions sufficient to restore the differentiation potential of the cells; thereby restoring the differentiation potential of the cells. The cells can be stem cells, primary cells, or bone marrow cells.

The invention also features a cell culture composition which includes a denatured polymeric matrix (e.g., denatured type I collagen, denatured type I collagen of between 0.1 mg/ml and 5 mg/ml, denatured type I collagen of 0.5 mg/ml, or denatured type I collagen of 0.3 mg/ml). The polymer can be type I collagen that has been denatured at 50° C. for 12 hours. The collagen matrix can be generated by evaporation of a denatured type I collagen solution at a concentration between 0.1 and 5 mg/ml (e.g., 0.3 mg/ml, 0.5 mg/ml) in a tissue culture dish. The cell culture compositions can be included in a kit for carrying out the methods described herein (e.g., methods of preserving one or more cellular functions that are characteristic of cells in a non-senescent state, where the one or more cellular functions are lost in cells that are in a senescent state; methods of restoring one or more cellular functions that are characteristic of cells in a non-senescent state, where the one or more cellular functions are lost in cells that are in a senescent state; methods of preserving cells in a non-senescent state; methods of restoring cells to a non-senescent state; methods of preserving the plasticity of cells; methods of restoring the plasticity of cells; methods of preserving the differentiation potential of cells; methods of restoring the differentiation potential of cells). Such a kit can also include packaging components and instructions for use.

The invention also features kits for carrying out the methods of the invention (e.g., methods of preserving one or more cellular functions that are characteristic of cells in a non-senescent state, where the one or more cellular functions are lost in cells that are in a senescent state; methods of restoring one or more cellular functions that are characteristic of cells in a non-senescent state, where the one or more cellular functions are lost in cells that are in a senescent state; methods of preserving cells in a non-senescent state; methods of restoring cells to a non-senescent state; methods of preserving the plasticity of cells; methods of restoring the plasticity of cells; methods of preserving the differentiation potential of cells; methods of restoring the differentiation potential of cells), where the kits include a matrix of denatured polymer (e.g., denatured type I collagen, denatured type I collagen of between 0.1 mg/ml and 5 mg/ml, denatured type I collagen of 0.5 mg/ml, or denatured type I collagen of 0.3 mg/ml), packaging compenents, and optionally, instructions for use.

The aged or aging cells are grown on a matrix of a disorganized biocompatible polymer, which causes the cells to exhibit cellular functions and characteristics that are normally associated with younger cells. Such cellular functions and characteristics, if lost in the aged cells, are regained, and if not yet lost, are maintained. Likewise, cellular functions and characteristics that are normally associated with aged or aging cells are lost when such cells are grown on the matrix as described herein.

The matrix should be a biocompatible polymer. A “biocompatible polymer” is a polymer (i.e., a substance that is made substantially (i.e., 95% or greater) of a repeating subunit molecule) that is “biocompatible”, that is, when introduced into the body of an organism, or placed in contact with cells in vitro, the polymer has no significant adverse effects on normal biological functions of the cells with which it is in contact (either in a tissue or organism or in vitro).

The biocompatible polymer can be a fibrous protein, a polyester, or a polysaccharide. The matrix can also be formed of more than one fibrous protein, more than one polyester, or more than one polysaccharide. The matrix can also be formed of a combination such polymers.

A “fibrous protein” is a protein with a highly repetitive amino acid sequence. This repetitive sequence leads to secondary structures (e.g., helices, sheets, etc.) that are characteristic of the protein in its native state. Collagens and silks are two different examples of this class of polymer. Collagen, for example, forms triple helices, and silks (fibroins) form beta sheets. Other examples of fibrous proteins include, but are not limited to, keratins, tubulins, actins, elastins, myosins.

Polyesters are also appropriate polymers to be used in the invention. A “polyester” is a polymer characterized by an ester chemical bond between the monomer units. This bond is chemically hydrolyzable or enzymatically hydrolyzable, and thus the polymers are biodegradable (i.e., bioerodible, biocompatible). Examples of polyesters include, but are not limited to, polycaprolactone, polylactic acid, polyglycolic acid, polynucleic acids, polyhydroxyalkanoates.

Polysaccharides are also useful for producing a matrix of the invention. “Polysaccharides” form a heterogeneous group of polymers of different length and composition. They are polymers constructed from monosaccharide residues (sugar monomer units) that are linked by glycosidic bonds. A polysaccharide may consist of one type of monomer (i.e., be a homopolymer) or may consist of several types of monomers (i.e., be a heteropolymer). Examples of polysaccharides include, but are not limited to, alginate, chitosan, chitin, gellan, pullulan, cellulose, hyaluronic acid, starches (e.g., amylose, amylopectin, pectin), glycogen, glycosaminoglycan (e.g., hyaluronate, chondroitin, heparin), dextrin, inulin, mannan, chitin. Alginate is a polysaccharide that consists exclusively of uronic acids: mannuronic acid and beta-L-glucuronic acid in changing ratios and of small amounts of beta-D-glucuronic acid. Both homo- and heteropolymeric forms exist. Alginates have a high affinity for divalent cations (e.g., calcium, strontium, barium, magnesium) and have a tendency to form well-defined gel networks.

Lignin, glutenin, polyhydroxyalkanoates, polyisoprenoids, arabinoxylans, polyamides, polyimides, polyurethanes, polyethylene, polypropylene, polyvinylchloride and polystyrene are also useful in the invention to the extent that they are biocompatible.

Polymers useful in forming a matrix of the invention are those biocompatible polymers which normally exhibit a higher structural order, and which, in the course of practicing the invention, are denatured or disorganized.

By “denatured” or “disordered” is meant that the polymer (after treatment intended to cause denaturation or disorganization) lacks clearly definable structural features upon characterization, e.g., no longer possesses previously-held secondary, tertiary or quaternary structure, or crystallinity, etc.

“One or more cellular functions that are characteristic of cells in a non-senescent state” refers to those functions exhibited by cells that are vigorous and non-apoptotic, and includes the expression of one or more genes and proteins, where such expression is lost or reduced in senescent and apoptotic cells. Such genes and proteins include β-galactosidase, hsp70, and other stress response-related genes, expression of cFos, expression of SA-β-galactisidase, lipofuscin accumulation, ornithine decarboxilase and thymidine kinase activities, levels of lamp2 lysozomal receptor, length of telomeres, telomerase activity, level of protein oxidation, DNA integrity (such as single-stranded breaks), RNA structure (such as the length of polyA tails), number of copies of certain genes (such as ribosomal genes), number of mitochondria or other organelles, evaluation of cell morphology, as well as any additional aging marker or assay to be identified. The term also refers to genes and proteins the activity of which increases as a result of aging, e.g., as with genes associated with apoptosis.

The term also refers to morphological changes in cells as they age, for instance, as cells approach senescence, they become poorly defined morphologically, and become larger, occupying an area two or more times that occupied by younger cells. Such functions and proteins include the ability of cells to resist and recover from stresses, e.g., the ability to express stress-related proteins, such as heat shock proteins, e.g., hsp70. Such functions and proteins can vary according to the type of cells, e.g., in BMSCs, such functions would include the ability of the cells to deposit calcium, and such proteins would include the expression of alkaline phosphatase.

Because age effects are often seen at forty population doublings or more, and because “young” cells are usually defined as those that have gone through twenty or fewer population doublings, the level of expression of a gene at twenty population doublings is taken herein to represent the level of “baseline” expression that is seen in young cells. For those cellular functions that decrease with advanced age, the invention seeks to restore those cellular functions to a level of 50% or greater relative to “baseline.” For those cellular functions that increase with advanced age, the invention seeks to reduce those cellular functions to a level of 50% or less relative to “baseline.”

The term also refers to cell “plasticity,” e.g., the ability of an undifferentiated cell to remain in an undifferentiated state beyond the time at which it would normally have differentiated, and also differentiation potential, which is the ability of an undifferentiated cell to differentiate at a time beyond which it would normally have lost the ability to do so. For instance if, in a population of undifferentiated cells of a particular type and not maintained as described herein, some of the cells would normally begin differentiating at PD8, most differentiate around PD10, and substantially all of the cells are differentiated around PD12, then maintenance of cell plasticity, as the term is used herein, would result in the majority of the cells remaining undifferentiated at a PD that is statistically significantly later than that normally seen in cells of the same type which are not maintained as described herein. If, in a population of undifferentiated cells of a particular type and not maintained as described herein, some of the cells would normally begin losing their potential to differentiate at PD16, most lose such potential around PD18, and substantially all of the cells have lost this potential around PD20, then maintenance of cell plasticity, as the term is used herein, would result in the majority of the cells remaining undifferentiated, yet retaining their potential to differentiate, at a PD that is statistically significantly later than that normally seen in cells of the same type which are not maintained as described herein.

“Cells in a non-senescent state” are cells that do not exhibit characteristics normally associated with apoptosis and the various stages of cells death, such as loss of or reduction in hsp70 expression, loss of or reduction in β-galactosidase expression, hsp70, other stress response-related genes, expression of cFos, expression of SA-β-galactisidase, lipofuscin accumulation, ornithine decarboxilase and thymidine kinase activities, levels of lamp2 lysozomal receptor, length of telomeres, telomerase activity, level of protein oxidation, DNA integrity (such as single-stranded breaks), RNA structure (such as the length of polyA tails), number of copies of certain genes (such as ribosomal genes), number of mitochondria or other organelles, evaluation of cell morphology, as well as any additional aging marker or assay to be identified.

The cells can be a type of stem cell (e.g., embryonic, bone marrow, adipose, skin, amnionic fluid, etc.), a primary differentiated cell isolate (e.g., fibroblast, osteoblast, chondrocyte, etc.), or any secondary cell line isolate.

By “matrix of denatured type I collagen” is meant a growth medium that is made primarily of type I collagen, that is, is made up substantially entirely of type I collagen, that has been substantially completely denatured, e.g., by heating at 50° C. for 12 hours. “Collagen Type I”, like other collagens, is an insoluble, extracellular, glycoprotein, and it is one of the main components of connective tissue, such as bones, ligaments and tendons. For example, collagen (Roche, Basel, Switzerland, cat. #1179179) from rat tail tendon is dissolved at 5 mg/ml in 0.1% acetic acid and denatured by incubation at 50° C. for 12 hours (Payne and Veis, 1988). To prepare the films, 1.5 ml of collagen solution is added to a 35 mm tissue culture dish (washed with tissue culture medium prior to use) and dried under vacuum, as is described in Example 2, below.

In experiments involving BMSC cells, 1.5 ml of 0.5 mg/ml collagen solutions were added to 35 mm tissue culture dish (Coming Incorporated Life Sciences, Acton, Mass., USA) and dried under vacuum. When larger dishes were used, the same ratio of collagen volume per dish area was applied. Control dishes were treated similarly but with the solution of 0.1% acetic acid. Dishes were washed with tissue culture medium prior to use.

“Culturing the cells” refers to the range of growth conditions (e.g., temperature, humidity, etc.) normally tolerated by the cells.

“Preserving cells in a non-senescent state” means preventing the onset in cells of one or more of those characteristics normally associated with apoptosis and cell death, e.g., preserving the morphology and/or the size of non-senescent cells, the level of hsp70 expression and/or β-galactosidase expression, relative to senescent cells. In general, the expression of hsp70 and/or β-galactosidase should be restored to 50% or more of that of young (≦PD20) cells.

By “plasticity” is meant the ability of undifferentiated cells to remain in an undifferentiated state. In general, the number of cells that spontaneously differentiate should be 50% or less than that seen in control cells. By “differentiation potential”, or “potential for differentiation”, or in the case of bone marrow stromal cells, “potential for osteogenic differentiation” is meant the ability of undifferentiated cells to differentiate. In general, at least about 50% of the cells should retain the ability of differentiate after 20 doublings.

The substrate used can be any known polymeric matrix such as, but not limited to, collagen, silk, alginic acid, polyesters, polylactic acid or copolymers with glycolic acid, as well as any additional polymeric matrix. In general, the substrate is prepared by being “disorganized”, that is, the substrate is treated so that it is reduced to an organizational level below its native state, e.g., so that it has lost its secondary structure. Proteins such as collagen, for instance, can be denatured by simple boiling. Other polymeric matrices are treated in ways known to those of ordinary skill familiar with their properties, so as to reduce the organizational state of the polymer. The organizational state of the polymer after treatment can be assessed by methods known to those of ordinary skill who are familiar with the polymers. Such methods include, but are not limited to, circular dichroism spectroscopy (e.g., for collagen), fourier transform infrared spectroscopy (e.g., for silk), gel formation vs. absence of gelation in the presence of calcium ions (e.g., for alginic acid), X-ray analysis for degree of crystallinity (e.g., for polylactic acid or copolymers with glycolic acid). In general, about 50% or more of the polymer should be disorganized.

Applications of the invention include cell rejuvenation for purposes of cloning and reduction of the rate of aging during expansion of stem cells for purposes of tissue engineering. The construction of a tissue, including sometimes massive in vitro expansions of relatively few stem cells, can involve a substantial number of cell divisions, resulting in cells becoming “old” by the time of implantation.

### BRIEF DESCRIPTION OF THE DRAWINGS

FIGS. 1A-1F are a set of six photographs showing that growth of IMR90 cells on a denatured collagen matrix reverses aging-related morphological changes. Young (PD30; population doubling 30) cells (“A”), and aged (PD64) cells (“B”) were grown for six days on the following matrices: FIG. 1A, FIG. 1B: tissue culture dishes; FIG. 1C: film of 5 mg/ml denatured collagen; FIG. 1D: film of 3 mg/ml denatured collagen; FIG. 1E: film of 0.5 mg/ml denatured collagen; FIG. 1F: film of 0.5 mg/ml native collagen. Preparation of collagen films and treatment of control dishes are described below. Pictures were taken using Zeiss Axiovert S100 microscope at magnification ×150, and Sony Exwave HAD 3CCD color video camera.

FIGS. 2A-2D are a set of four photographs showing that prolonged growth of young IMR90 cells on a denatured collagen matrix decreases the rate of aging-related morphological changes. Young (PD24) cells were maintained (with the exception of FIG. 2A, which represents time zero) for 10 passages (approximately 30PD) on the following matrices: FIG. 2A, FIG. 2B: tissue culture dishes; FIG. 2C: film of 0.5 mg/ml denatured collagen; FIG. 2D: film of 0.5 mg/ml native collagen. Dishes were prepared and treated as described below. For each passage, cells were trypsinized, diluted 8-10 times, plated on fresh dishes and allowed to reach confluence prior to next passage. Pictures were taken using Zeiss Axiovert S100 microscope at magnification ×150, and Sony Exwave HAD 3CCD color video camera.

FIGS. 3A-3G are a set of six photographs and one graph showing that growth on a denatured collagen matrix results in the cessation of β-galactosidase expression in aged IMR90 cells. FIGS. 3A-3F: all procedures and conditions were the same as described in the legend to FIG. 1. Staining for β-galactosidase was carried our as described by Dimri et al. (1995), and in Example 3, below. Pictures were taken using Zeiss Axiovert S100 microscope at magnification ×150, and Sony Exwave HAD 3CCD color video camera. FIG. 3G is a quantitative representation of the percentage of β-galactosidase-positive cells; “A” to “F” correspond to the treatments in FIGS. 3A-3F. “A”: cells, PD30, grown on tissue culture dish; “B”: cells, PD64, grown on tissue culture dish; “C”: cells, PD64, grown on film of 5 mg/ml denatured collagen; “D”: cells, PD64, grown on film of 3 mg/ml denatured collagen; “E”: cells, PD64, grown on film of 0.5 mg/ml denatured collagen; “F”: cells, PD64, grown on film of 0.5 mg/ml native collagen. Each point represents the mean and standard deviation of triplicate determinations (1000 cells per determination).

FIGS. 4A-4E are a set of four photographs and one graph showing that prolonged growth of young IMR90 cells on a denatured collagen matrix results in the decreased β-galactosidase. FIGS. 4A-4D: all procedures and conditions were the same as described in the description of FIG. 2, above. Staining for β-galactosidase was carried our as described by Dimri et al. (1995), and in Example 3, below. Pictures were taken using Zeiss Axiovert S100 microscope at magnification ×150, and Sony Exwave HAD 3CCD color video camera. FIG. 4E is a quantitative representation of the percentage of β-galactosidase-positive cells; “A”-“E” correspond to the treatments in FIGS. 4A-4D. “A”: cells, PD24, at time zero; “B”: cells, PD54, maintained in tissue culture dishes; “C”: cells, PD54, maintained on films of 0.5 mg/ml denatured collagen; “D”: cells, PD54, maintained on films of 0.5 mg/ml native collagen. Each point represents the mean and standard deviation of triplicate determinations (1000 cells per determination).

FIGS. 5A-5B are an immunoblot and a graph, respectively, showing that growth of aged IMR90 cells results in the reduction of oxidation in a subset of cellular proteins. FIG. 5A: aged (PD64) cells were grown on different matrices. Dishes were prepared and treated as described below. After six days cells were collected, samples were prepared and analyzed by gel electrophoresis and immunoblotting as detailed below. Lane 1: tissue culture dishes; lane 2: film of 5 mg/ml denatured collagen; lane 3: film of 0.5 mg/ml native collagen; lane 4: film of 0.5 mg/ml denatured collagen. FIG. 5B shows quantitative representation of relative oxidation at different conditions of a band indicated by an arrow and marked “I” in FIG. 5A. For the reason of comparison, relative oxidation of a largely unchanged subset of proteins marked by a bracket and designated “II” in FIG. 5A is also shown. 1-4: same as in FIG. 5A. Each point represents the mean and standard deviation of triplicate determinations in separate experiments.

FIGS. 6A and 6B are an immunoblot and a graph, respectively, showing that growth of aged IMR90 cells on a denatured collagen matrix results in the restoration of Hsp70 expression in response to stress. FIG. 6A: young, PD30, cells (lanes 1, 2), and aged, PD64, cells (lanes 3-14) were grown on the following matrices: Lanes 1-4: tissue culture dishes; lanes 5, 6: film of 3 mg/ml denatured collagen; lanes 7, 8: film of 0.5 mg/ml native collagen; lanes 9, 10: film of 1 mg/ml denatured collagen; lanes 11, 12: film of 0.5 mg/ml denatured collagen; lanes 13, 14: film of 0.3 mg/ml denatured collagen. Dishes were prepared and treated as described below. After six days half of the dishes (even-numbered lanes) were subjected to a thermal stress (44° C., 30 min), while the other half (odd-numbered lanes) served as control. Six hours following the thermal stress (to allow for potential accumulation of stress-induced Hsp70), cells were collected and analyzed for the presence of Hsp70 by gel electrophoresis and immunoblotting as described in Example 4 below. FIG. 6B is a quantitative representation of relative expression of Hsp70 in: 1: young stressed cells grown in tissue culture dish; 2: aged stressed cells grown in tissue culture dish; 3: aged stressed cells grown on film of 3 mg/ml denatured collagen; 4: aged stressed cells grown on film of 0.5 mg/ml native collagen; 5: aged stressed cells grown on film of 1 mg/ml denatured collagen; 6: aged stressed cells grown on film of 0.5 mg/ml denatured collagen; 7: aged stressed cells grown on film of 0.3 mg/ml denatured collagen. Each point represents the mean and standard deviation of triplicate determinations in separate experiments.

FIG. 7 is a graph showing the determination of transition temperature for collagen I. Temperature (in ° C.) is shown on the x-axis, and the ratio of unfolded to folded protein is shown on the y-axis. Thermotransition for collagen I was determined using circular dichroism profiles obtained on a Jasko J-710 spectropolarimeter.

FIG. 8 is a photo of a polyacrylamide gel, and shows the gel analysis of native and denatured collagen. Samples of native collagen and collagen were denatured by treatment at 50° C. for different time periods, and were resolved on a denaturing 7.5% polyacrylamide tris-acetate gel and stained with Comassie blue. Lane 1: MW standards; 2: native collagen; 3: two hours of thermal treatment; 4: four hours of thermal treatment; 5: eight hours of thermal treatment; 6: 12 hours of thermal treatment; 7: 16 hours of thermal treatment; 8: 20 hours of thermal treatment; 9: 24 hours of thermal treatment.

FIGS. 9A, 9B and 9C are a set of three photomicrographs, showing that growth of BMSC cells on a denatured collagen matrix reduces the rate of cellular aging-related morphological changes. Passage 2 BMSCs were maintained (with the exception of FIG. 9A, which represents time zero) through passage 11 on the following matrices. FIG. 9A, 9B: tissue culture dishes; FIG. 9C: film of 0.5 mg/ml denatured collagen; Dishes were prepared and treated as described in the Examples below. For each passage, cells were trypsinized, diluted 8-10 times, plated on fresh dishes and allowed to reach about 90% confluence prior to next passage. Pictures were taken using Zeiss Axiovert S100 microscope at magnification ×150, and Sony Exwave HAD 3CCD color video camera.

FIG. 10 is a graph showing that prolonged cultivation on a denatured collagen matrix results in the retention of the expression of early osteogenic marker, alkaline phosphatase, in response to OS treatment. BMSCs, grown either on tissue culture plastic or on collagen matrix (as indicated in the Figure), were treated for 7 days with osteogenic stimulants (+OS) or used as untreated controls (−OS). Alkaline phosphatase activity was determined as described in Example 6, below. Each point represents the mean and standard deviation of independent triplicate determinations. Young cells: passage 2 BMSCs; aged cells: passage 11 cells grown on tissue culture plastic and passage 14 cells cultivated on a denatured collagen matrix. Different numbers of passages were used due to faster growth of cells on the collagen matrix.

FIG. 11 is a graph showing that prolonged cultivation on a denatured collagen matrix preserves the ability of BMSCs to mineralize the extracellular matrix in response to OS treatment. BMSCs, grown either on tissue culture plastic or on collagen matrix (as indicated in the Figure), were treated for 14 days with osteogenic stimulants (+OS) or used as untreated controls (−OS). Extracellular calcium deposition was measured as described in Example 7, below. Each point represents the mean and standard deviation of independent triplicate determinations. Double asterisks indicate non-detectable levels of calcium. Young cells: passage 2 BMSCs; aged cells: passage 11 cells grown on tissue culture plastic and passage 14 cells cultivated on a denatured collagen matrix. Different numbers of passages are due to faster growth of cells on the collagen matrix.

FIG. 12 is a photograph showing that prolonged cultivation on a denatured collagen matrix preserves the BMSC's potential to express late osteogenic-specific gene, bone sialoprotein (“BSP”), in response to OS treatment. BMSCs, grown through passage 11 on tissue culture plastic and cultivated through passage 14 on a denatured collagen matrix, were either used as untreated controls or treated for 14 days with OS (osteogenic stimulants, see Examples, below). Different numbers of passages were used due to faster growth of cells on the collagen matrix. Total RNA was obtained, BSP transcripts were amplified by RT-PCR and analyzed as described in Example 8, below. 1: untreated cells grown on tissue culture plastic; 2: treated cells grown on tissue culture plastic; 3: untreated cells grown on collagen matrix; 4: treated cells grown on collagen matrix. The expression of BSP was normalized to housekeeping GAPDH (“GAPDH”).

FIG. 13 is a graph showing BMSC HSP70 induction in response to stress. Expression of BMSC HSP70 mRNA was measured by RT-PCR as described in Example 17, below. BMSCs, grown either on tissue culture plastic or on collagen matrix (as indicated in the Figure), were untreated or subject to heat shock (44° C. for 45 minutes). Each point represents the mean and standard deviation of independent triplicate determinations. Young cells: early passage cells (thawed aliquot of passage one BMSC cells); old cells: late passage cells (passage 8 cells started from a thawed aliquot of passage one cells).

FIG. 14 is a graph showing alkaline phosphatase activity in response to the presence or absence of serum when either grown on plastic or maintained on denatured collagen matrix.

FIG. 14 shows that (a) growth on denatured collagen matrix preserves the potential for OS-mediated alkaline phosphatase expression in ex vivo expanded BMSCs, and (b) the absence of serum significantly diminishes but does not eliminate the effect of collagen matrix.

Alkaline phosphatase activity was calculated after measuring the absorbance of p-nitrophenol product, nmol/20 min/10⁵ cells, as described in Example 6, below. Each point represents the mean and standard deviation of independent triplicate determinations. Double asterisks indicate non-detectable levels of p-nitrophenol. BMSCs, grown either on tissue culture plastic or on collagen matrix (as indicated in the Figure), were treated for 10 days with osteogenic stimulants (+OS) or used as untreated controls (−OS). Young cells: early passage cells (thawed aliquot of passage one BMSC cells); aged cells: late passage cells (passage 8 cells started from a thawed aliquot of passage one cells). “Aged Plastic, −OS/Coll” refers to BMSC cells aged on plastic and induced on collagen in the absence of OS. “Aged Plastic, +OS/Coll” refers to BMSC cells aged on plastic and induced on collagen in the presence of OS.

FIG. 15 is a graph showing the ability of early and late passage BMSCs, grown on plastic or on a denatured collagen matrix either in the presence or in the absence of serum, to deposit, in response to OS treatment, extracellular calcium as an indicator of later stage osteogenic potential. Extracellular calcium deposition (micrograms/dish) was measured as described in Example 7, below. Each point represents the mean and standard deviation of independent triplicate determinations. Double asterisks indicate non-detectable levels of calcium. BMSCs, grown either on tissue culture plastic or on collagen matrix (as indicated in the Figure), were treated for 14 days with osteogenic stimulants (+OS) or used as untreated controls (−OS). Young cells: early passage cells (thawed aliquot of passage one BMSC cells); aged cells: late passage cells (passage 8 cells started from a thawed aliquot of passage one cells). “Aged Plastic, −OS/Coll” refers to BMSC cells aged on plastic and induced on collagen in the absence of OS. “Aged Plastic, +OS/Coll” refers to BMSC cells aged on plastic and induced on collagen in the presence of OS.

### DETAILED DESCRIPTION

The present invention relates to methods and compositions for growing young cells that reduces and/or reverses age-related processes that would otherwise occur in those cells. The invention is based on the discovery that growth of primary human cells on certain collagen matrices results in “rejuvenation” of aged cells and appears to significantly reduce the rate of aging in young human cells.

To use a polymer according to the present invention, a polymer should be chosen so that, when denatured or disorganized, it will still form a solid, a semi-solid, or a gel, so that cells can be grown upon it. The polymer should be biocompatible, so that it will not interfere with the normal biological functions and processes of the cells. Many such polymers are known in the pharmaceutical and medical arts, and others can readily be tested for biocompatibility.

The polymer is then treated so as to denature it (in the case of proteins), so as to “disorganize” it. This can be done with heat, pressure, chemicals, irradiation, or other means, so long as the treatment does not interfere with the biocompatibility of the polymer. The treatment should alter the polymer so that it is reduced to a lower organizational level, that is, it is changed from a higher structural order to a lower structural order. By “higher structural order” is meant the different length scales of interaction that can be distinguished based upon the structural characterization of the polymers, e.g., networked gel, triple helices, crystalline domains, e.g., the “higher structural order” of native collagen is exhibited in its ability to form triple helices, and the “higher structural order” of silk is exhibited in its ability to for beta sheets. The “denatured” or “disorganized” forms of these polymers will be lacking such clearly definable structural features upon characterization, that is, the polymers will no longer have the same secondary or tertiary structures as the native forms of those polymers. Collagen, for example, will be denatured so that it no longer possesses helical structure. Silk should be denatured so that it is no longer in the form of beta sheets. Polyesters will be denatured so that they are amorphous rather than crystalline.

After treatment to denature or disorganize the polymer(s), the organizational state of the polymer can be assessed by methods known to those of ordinary skill who are familiar with the characteristics of the polymers. Such methods include, but are not limited to, circular dichroism spectroscopy (e.g., for collagen), fourier transform infrared spectroscopy (e.g., for silk), gel formation vs. absence of gelation in the presence of calcium ions (e.g., for alginic acid), X-ray analysis for degree of crystallinity (e.g., for polylactic acid or copolymers with glycolic acid). In general, 50% or more of the polymer should be disorganized.

The disorganized polymer (or mixture of polymers) can then be used as a matrix for growing cells. In the case of polymers that can be denatured by simple boiling or other aqueous treatment, the polymer can be diluted. The level of dilution should not be so great that the polymer no longer forms a solid, semi-solid or gel-like surface for the cells to grow upon. As shown herein, for instance, denatured collagen at both 0.3 mg/ml and 0.5 mg/ml has a regenerative effect on aged cells grown upon this matrix. The polymer should be handled after treatment so as to prevent the polymer from regaining its native organizational complexity.

A. Denatured Collagen Matrix

Collagen Type I, a key extracellular matrix protein, was assessed for its potential impact on the process of cellular aging. To make the matrix, collagen (Roche, Basel, Switzerland, cat. #1179179) from rat tail tendon is dissolved at 5 mg/ml in 0.1% acetic acid and denatured by incubation at 50° C. for 12 hours (Payne and Veis, 1988). To prepare the films, 1.5 ml of collagen solution is added to a 35 mm tissue culture dish (washed with tissue culture medium prior to use) and dried under vacuum, as is described in Example 2, below.

Collagen matrices of 5 mg/ml to 3 mg/ml were found to have no beneficial effect on cells grown upon them. Matrices made of native collagen (i.e., not denatured as is described herein) at the same concentration, however, resulted in cell death. Beneficial results, in terms of preservation of cell function, morphology and gene expression, were seen when cells were grown on denatured collagen matrices of 1 mg/ml, were more beneficial at 0.5 mg/ml, and were even more beneficial when the cells were grown on denatured collagen matrices of 0.3 mg/ml. Lower concentrations were not tested, however, due to incomplete coverage of the culture dish, that is, when dried under vaccuum, the film pulled back, leaving bare spots and holes in the film. Concentrations lower than 0.3 mg/ml can be used to make the denatured collagen matrices as described herein, so long as the film is prevented from pulling back during drying.

Other polymeric substrates can also be used, e.g., silk, alginic acid, polyesters, polylactic acid or copolymers with glycolic acid, as well as any additional polymeric matrix. In general, the substrate is prepared by being “disorganizing”, that is, the substrate is treated so that it is reduced to an organizational level below its native state, e.g., so that it has lost its secondary structure. Proteins such as collagen, for instance, can be denatured by simple boiling. Other polymeric matrices are treated in ways known to those of ordinary skill familiar with their properties, so as to reduce the organizational state of the polymer. The organizational state of the polymer after treatment can be assessed by methods known to those of ordinary skill who are familiar with the polymers. Such method include, but are not limited to, circular dichroism spectroscopy (e.g., for collagen), fourier transform infrared spectroscopy (e.g., for silk), gel formation vs. absence of gelation in the presence of calcium ions (e.g., for alginic acid), X-ray analysis for degree of crystallinity (e.g., for polylactic acid or copolymers with glycolic acid). Iin general, about 50% or more of the polymer should be disorganized. The polymer chosen to be used should be such that when dried, continuous surface is formed upon which to grow the cells.

B. Cells Useful in the Invention

The cells can be a type of stem cell (e.g., embryonic, bone marrow, adipose, skin, amnionic fluid, etc.), a primary differentiated cell isolate (e.g., fibroblast, osteoblast, chondrocyte, etc.), or any secondary cell line isolate. In general, any cell capable of undergoing division and capable on growing on a solid matrix can be used in the invention.

C. Culture Conditions

Once seeded onto the disorganized substrate, the cells are grown according to those methods appropriate for and specific to the cells. Such methods are known to those of ordinary skill in the art of growing such cells.

D. Preserving Cell Functions

The cells, when grown on the disorganized substrate, show reversal of one or more aging-associated or apoptosis-associated cellular functions, and/or maintenance of one or more cellular functions that are characteristic of non-aged cells. Such cellular functions include, but are not necessarily limited to, expression of β-galactosidase, hsp70, and other stress response-related genes, expression of cFos, expression of SA-β-galactisidase, lipofuscin accumulation, ornithine decarboxilase and thymidine kinase activities, levels of lamp2 lysozomal receptor, length of telomeres, telomerase activity, level of protein oxidation, DNA integrity (such as single-stranded breaks), RNA structure (such as the length of polyA tails), number of copies of certain genes (such as ribosomal genes), number of mitochondria or other organelles, evaluation of cell morphology, as well as any additional aging marker or assay to be identified. The expression of one or more such age-related genes is reduced by at least 50% in senescent cells treated according to the invention, relative to equivalent untreated cells. Likewise, for cellular functions the activity of which decreases with age, the expression of such genes is increased to 50% or greater, relative to the expression seen in cells not treated according to the invention.

As shown herein, growth on a denatured collagen matrices reversed in aged cells not only the attenuation of Hsp70 expression but also other aging-related processes, such as β-galactosidase expression, increase in protein oxidation and changes in cell morphology. When BMSCs are grown on a denatured collagen matrix, the rate of morphological changes is significantly reduced, and results in a dramatic increase in the retention by aged cells of the potential to express osteogenic-specific functions such as osteogenic potential, and to express specific markers upon treatment with osteogenic stimulants. Moreover, growth on a matrix of denatured collagen appeared to reduce the rate of aging in young cells. Understanding the nature of collagen matrix-mediated cellular rejuvenation also suggests approaches for interfering with organismic aging.

The results described herein show that growth on a denatured collagen matrix reverse not only the attenuation of Hsp70 expression but also other aging-related processes, including changes in cell morphology, in aged cells, and reduce the rate of aging in young cells. The inverse proportionality of the effects observed relative to collagen concentration indicates that matrix topography can play a role in eliciting cellular responses described above. The invention utilizes collagen matrix-mediated cellular rejuvenation.

As shown herein, growth of primary human fibroblasts on a denatured collagen within a certain range of concentrations leads to the reduction of the rate of cellular aging, and growth of BMSCs on denatured collagen matrices results in a drastically increased the retention of osteogenic potential during prolonged cultivation.

A reduction in the rate of cellular aging translates into preservation of cellular functions and potentials, among them the potential of BMSCs to undergo, when properly stimulated, osteogenic differentiation. Growth of BMSCs on a denatured collagen matrix significantly reduced the rate of morphological changes, indicative of the reduction in the rate of cellular aging. The degree of increase in alkaline phosphatase activity were similar in cells grown on plastic and on the collagen matrix. Importantly, whereas only a marginal increase in alkaline phosphatase activity was observed in OS-treated aged cells grown on plastic, enzyme levels in aged cells maintained on collagen matrix were comparable to those seen in young OS-treated cells. The effects observed were OS-dependent; growth on collagen matrix in the absence of OS induced neither substantial alkaline phosphatase activity nor any detectable calcium deposition by either young or aged cells.

The same trend, seen with alkaline phosphatase, was observed when the extracellular deposition of calcium was analyzed, with the exception that even in young OS-treated cells, the degree of mineralization was higher on collagen matrices. With OS-treated aged cells, while very little mineralization was seen on plastic surfaces, the extent of calcium deposition on collagen matrices was comparable with that seen with OS-treated young cells.

Similar results were obtained when the expression of the “late” osteogenic-specific marker, bone sialoprotein, was analyzed by RT-PCR. Whereas OS treatment induced very little, if any, expression of bone sialoprotein in aged cells grown on plastic, a substantial increase in the levels of BSP-specific transcripts was seen in OS-treated cells maintained on collagen matrices. These results show that growth on a denatured collagen matrix preserves the potential of BMSCs to undergo osteogenic differentiation.

Denatured collagen at 0.5 mg/ml was used to culture BMSCs. As described herein, growth on a denatured collagen matrices at a certain range of concentrations leads to the reversal of several aging-associated processes in aged cells, and to the reduction of the rate of aging in young cells. Native collagen was not only ineffective, but inhibited cell growth. Moreover, in study with human fibroblasts, high concentrations (e.g., 3 mg/ml to 5 mg/ml and up) of denatured collagen were also ineffective. The effects observed with fibroblasts became apparent with 1 mg/ml of denatured collagen, and intensified with the decrease in collagen concentration. The lowest concentration tested was 0.3 mg/ml. The concentration of denatured collagen used in the present study, 0.3 and 0.5 mg/ml, were effective.

As demonstrated herein, that prolonged cultivation of BMSCs on a denatured collagen matrix reduces the rate of cellular aging and preserves differentiation potential.

As shown in the Examples, below, growth on certain denatured collagen matrices leads to the reversal of several aging-associated processes in aged cells and to the reduction of the rate of aging in young cells.

### EXAMPLE 1

### Experimental Procedures: Cells

Primary human fibroblasts IMR90 were grown in MEM supplemented with 20% fetal bovine serum, nonessential amino acids and 2 mM glutamine. Cells were usually seeded at a density 5×10⁴ cells/ml (about 10% confluent) and maintained at 37° C. in an atmosphere of 95% air and 5% CO₂. Cultures were replated when cell density reached confluence. Experiments were carried out at 50%-70% confluence. Pictures of cells were taken using Zeiss Axiovert S100 microscope at magnification ×150, and Sony Exwave HAD 3CCD color video camera.

Human BMSCs were isolated from human bone marrow aspirates. The aspirates were obtained from consenting, non-smoking donors of 25 years of age (Clonetics-Poietics, Walkersville, Md., USA) were resuspended in Dulbecco's modified eagle medium (DMEM) supplemented with 10% fetal bovine serum (FBS), 0.1 mM nonessential amino acids, 100 U/ml penicillin and streptomycin, and 1 ng/ml basic fibroblast growth factor (bFGF), plated at 10 μl aspirate/cm² in tissue culture flasks, and and maintained at 37° C. in an atmosphere of 95% air and 5% CO₂. All tissue culture components were from Life Technologies (Rockville, Md., USA). After about 10 days in culture, BMSCs were selected on basis of their ability to adhere to the tissue culture plastic, and non-adherent hematopoietic cells were removed during medium replacement. Medium was changed twice per week thereafter. During cultivation, cultures were replated using 0.25% trypsin, 1 mM EDTA when cell density reached about 90% confluence. Passage 2 cells were frozen, and one aliquot was thawed for prolonged cultivation either on plastic or on collagen matrix. Osteogenic induction was initiated using osteogenic stimulants (OS) consisting of (final concentration) 100 nM Dexamethasone, 10 mM β-glycerophosphate, and 0.05 mM ascorbic acid (all from Sigma Chemical Company, St Louis, Mo., USA). For osteogenic induction experiments (below) the following cells were used: “young” cells—a thawed aliquot of passage 2 cells; “aged” cells—passage 11 cells cultivated on plastic, and passage 14 of the same cells maintained on collagen matrix (cells on collagen grew faster than on plastic). For osteogenic induction experiments young cells were seeded at 5×10³ cells/cm², both on plastic and collagen, aged cells on collagen were seeded at 8×10³ cells/cm² (to compensate for the faster growth of the young cells), and aged cells on plastic were used when they reached about 75% confluence (to compensate for their very slow growth). At day 14 of the experiments, all cultures were confluent with the exception of the aged cells on plastic which were slightly subconfluent. Osteogenic induction experiments were carried out in the absence of bFGF. Pictures of cells were taken using a Zeiss Axiovert S100 microscope at magnification ×150, and Sony Exwave HAD 3CCD color video camera.

### EXAMPLE 2

### Experimental Procedures: Preparation of Collagen Films

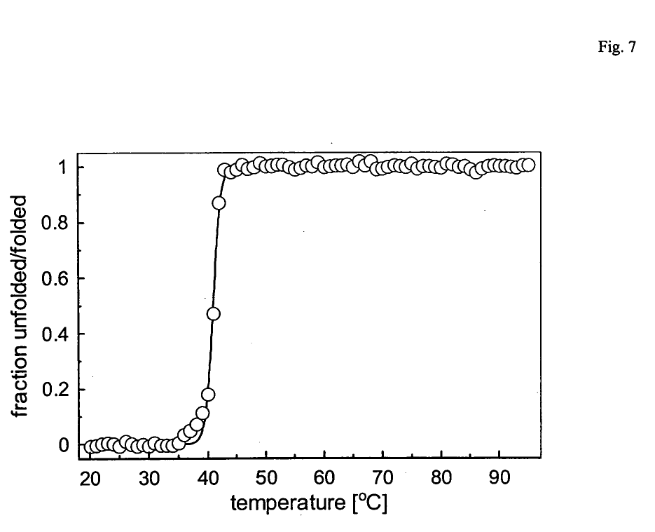

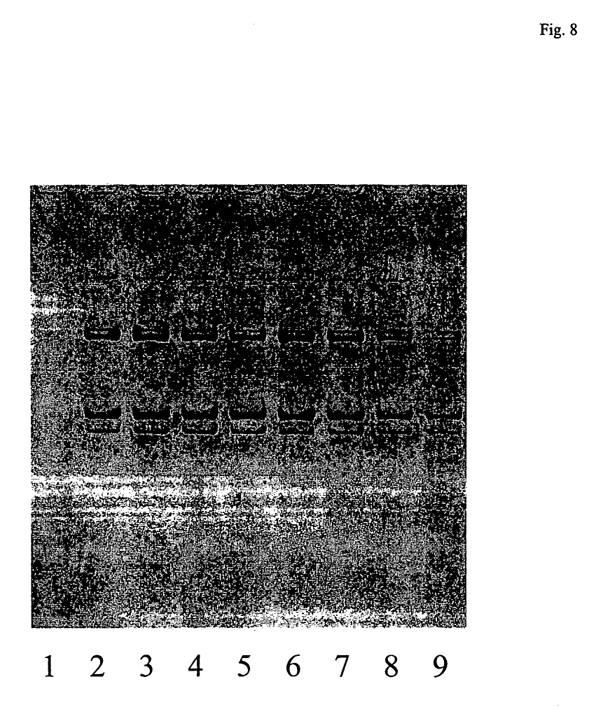

Collagen (Roche, Basel, Switzerland, cat. #1179179) was dissolved at 5 mg/ml in 0.1% acetic acid and denatured where indicated by incubation at 50° C. for 12 hours (Payne and Veis, 1988). These conditions were chosen based on complete denaturation confirmed using circular dichroism measurements to demonstrate a thermal transition at around 45° C. (FIG. 7). In addition, gel analysis of collagen denatured at 50° C. for various time periods showed that after 12 hours of treatment, the bulk of collagen remained intact in terms of molecular weight (FIG. 8).

To prepare films, 1.5 ml of collagen solutions of various concentrations was added to a 35 mm tissue culture dish (Corning Incorporated Life Sciences, Acton, Mass., USA) and dried under vacuum. Control dishes were treated similarly but with a solution of 0.1% acetic acid and no collagen. Dishes were washed with tissue culture medium prior to use.

In experiments involving BMSC cells, 1.5 ml of 0.5 mg/ml collagen solutions were added to 35 mm tissue culture dish (Coming Incorporated Life Sciences, Acton, Mass., USA) and dried under vacuum. When larger dishes were used, the same ratio of collagen volume per dish area was applied. Control dishes were treated similarly but with the solution of 0.1% acetic acid. Dishes were washed with tissue culture medium prior to use.

### EXAMPLE 3

### Experimental Procedures: β-Galactosidase Assay

Staining for β-galactosidase was carried out as described by Dimri et al., (1995). Briefly, cells were washed twice with PBS, fixed for 5 minutes at room temperature with 2% formaldehyde +0.2% glutaraldehyde, washed again twice in PBS, and incubated for 16 hours in staining solution (1 mg/ml X-gal in dimethylformamide, 40 mM citric acid/Na phosphate buffer, pH 6.0, 5 mM potassium ferrocyanide, 5 mM potassium ferricyanide, 5 M sodium chloride, 2 mM magnesium chloride) at 37° C. without CO₂.

### EXAMPLE 4

### Experimental Procedures: Thermal Stress and Hsp70 Detection

Cells were subjected to a thermal stress (44° C., 30 minutes) by floating parafilm-sealed dishes in a waterbath. Six hours following the thermal stress (to allow for potential accumulation of stress-induced Hsp70), cells were trypsinized, collected by centrifugation, lysed in a buffer containing 20 mM Tris-HCl pH 7.4, 50 mM NaCl, 2 mM EDTA, 1% Triton X100, 25 mM β-glycerophosphate, 10 mM NaF, 1 mM Na₃VO₄, and protease inhibitors (1 mM PMSF and 23 μg/ml each of aprotenin, pepstatin and leupeptin), resolved on SDS-7.5% Tris-acetate gel, transferred to nitrocellulose membrane, and immunoblotted with Hsp70-specific antibody (Spa 810) followed by incubation with secondary horseradish peroxidase-conjugated antibody. Bands were visualized by ECL using a reagent kit (ECL Plus) from Amersham Corp. (Arlington Heights, Ill., USA). To ensure equal loading on gels, relative protein concentrations in samples were determined using BioRad (Hercules, Calif., USA) protein assay reagent, and loading volumes were adjusted so that equal amounts of protein were loaded. Equal loading was ascertained by Ponceau staining of membranes immediately following Western transfer.

### EXAMPLE 5

### Protein Oxidation Assay

Cells were lysed as described above except that 50 mM DTT was added to lysis buffer to prevent the oxidation during derivatization procedure. The carbonyl groups in the protein side chains were derivatized to 2,4-dinitrophenylhydrazone (DNP-hydrazone) by reaction with 2,4-dinitrophenylhydrazine. Derivatization was carried out as described by the manufacturer of oxidation detection kit (Intergen Company, Purchase, N.Y., USA/Serologicals Corporation, Norcross, Ga., USA). Samples were resolved on SDS-7.5% Tris-acetate gel, transferred to nitrocellulose membrane, and immunoblotted with antibody to DNP-hydrazone followed by incubation with secondary horseradish peroxidase-conjugated antibodies. Bands were visualized by ECL using reagents kit (ECL plus) from Amersham Corp. (Arlington Heights, Ill., USA). To ensure equal loading on gels, relative protein concentrations in samples were determined using BioRad (Hercules, Calif., USA) protein assay reagent, and loading volumes were adjusted so that equal amounts of protein were loaded. Equal loading was ascertained by Ponceau staining of membranes immediately following Western transfer.

### EXAMPLE 6

### Alkaline Phoshatase Assay

To measure alkaline phospatase activity, cells (in triplicate cultures) were washed twice with PBS lacking calcium and magnesium, and resuspended in 0.25 IGEPAL CA630 (Sigma Chemical Company, St. Louis, Mo., USA). After centrifugatuion, an aliquot of supernatant was incubated with 5 mM p-nitrophenyl phosphate in 0.15 M 2-amino-2-methyl-1 -propanol, 1 mM MgCl₂, pH 10.5, at 37° C. for 20 minutes. Alkaline phosphatase activity was calculated after measuring the absorbance of p-nitrophenol product at 405 nm on a microplate reader and comparing it with known standards.

### EXAMPLE 7

### Extracellular Calcium Assay

Calcium assays were performed as described in Bruder et al., (1997), using calcium diagnostic kit #587 (Sigma Chemical Company, St. Louis, Mo., USA). Briefly, cells (in triplicate cultures) were washed twice with PBS lacking calcium and magnesium, and scraped off the dish in 0.5 M HCl. The calcium was extracted by shaking for 1 hour. After centrifugation at 1000 g, an aliquot of supernatant was used for calcium determination according to manufacturer's instructions. Absorbance of samples was measured at 575 nm on a microplate reader and compared with known standards.

### EXAMPLE 8

### RT-PCR Analysis of Bone Sialoprotein (BSP) and GAPDH

RT-PCR analysis of late osteogenic marker, bone sialoprotein (BSP), and of a housekeeping gene GAPDH, was carried out using the Access RT-PCR system (Promega, Madison, Wis., USA) in accordance with the manufacturer's instructions. Briefly, reaction mixtures consisted of 2 μl of total RNA (100 ng) combined with 31 μl distilled water, 10 μl of 5× AMV/Tfl Reaction Buffer, 1 μl of 10 mM dNTPs, 2 μl 25 mM MgSO₄, 1 μl of AMV reverse transcriptase (5 U/μl), 1 μl of Tfl DNA polymerase (5 U/μl), and 50 pmoles of the respective forward and reverse primers. All primers were designed with Primer Select software (Perkin-Elmer Applied Biosystems, Foster City, Calif., USA). Primers for bone sialoprotein and GAPDH were as follows: Bone sialoprotein forward primer=5′ AAG CAA TCA CCA AAA TGA AGA CT 3′ (SEQ ID NO:1); Bone sialoprotein reverse primer=5′ TGG AAA TCG TTT TAA ATG AGG ATA 3′ (SEQ ID NO:2). GAPDH forward primer=5′ GGG CAT CCT GGG CTA CAC TGA G 3′ (SEQ ID NO:3); GAPDH reverse primer=5′ GGC CCC TCC CCT CTT CAA G 3′ (SEQ ID NO:4). PCR reactions were performed using a PTC-100 thermocycler (MJ Research, Watertown, Mass., USA). With the exception of amplification temperature, the same reaction conditions were used for each primer set: first strand cDNA synthesis: 48° C. for 45 minutes, 94° C. for 2 minutes; second strand cDNA synthesis and PCR amplification: 94° C. for 30 seconds, amplification temperature for 1 minutes, 68° C. for 2 minutes; and final extension: 68° C. for 7 minutes. Forty amplification cycles were used for both markers and the amplification temperature for BSP was 53.8° C., and 58° C. for GAPDH. Total RNA was prepared using TRIzol (InVitrogen, San Diego, Calif., USA) procedure followed by RNeasy (Qiagen, Hilden, Germany) protocol to assure the absence of DNA in RNA preparations. PCR products were analyzed by electrophoresis on 2.2% agarose gels in 1×TBE along with molecular weight markers (100 bp DNA ladder, Gibco (Life Technologies, Gibco/BRL, Gaithersburg, Md., USA)) and visualized by ethidium bromide staining using a Fluoro-S Multilmager (Biorad, Hercules, Calif., USA). The expression of BSP was normalized to GAPDH.

### EXAMPLE 9

### Growth on a Denatured Collagen Matrix Confers to Aged Cells those Characteristics of Cells in a Non-Senescent State and Appears to Reduce the Rate of Aging in Young Cells

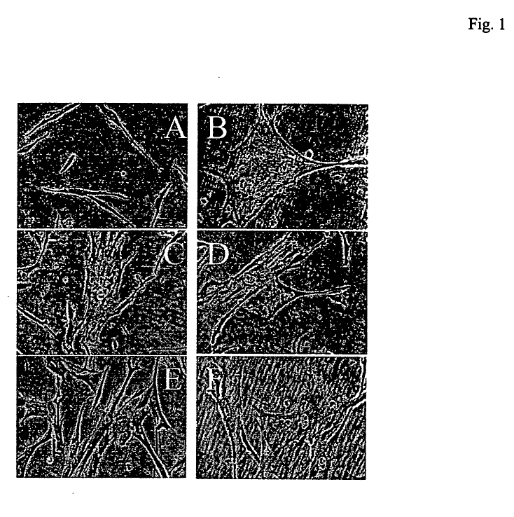

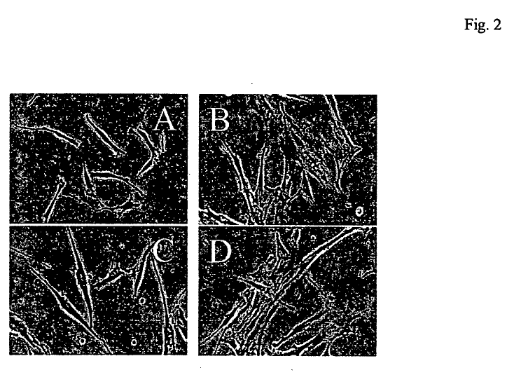

With increasing number of population doublings (PD5) in culture, IMR90 cells undergo substantial and well defined morphological changes. Slim and morphologically well organized young cells gradually increase in size, spread on the dish and send out numerous podia. When approaching senescence, aged cells (PD64) are poorly defined morphologically, and occupy an area several times that occupied by young cells. This is shown in FIGS. 1A and 2B, which are a pair or photographs of young, PD30 (population doubling 30) cells (FIG. 1A) and aged PD64 cells (FIG. 1B), grown for 6 days on tissue culture dishes.

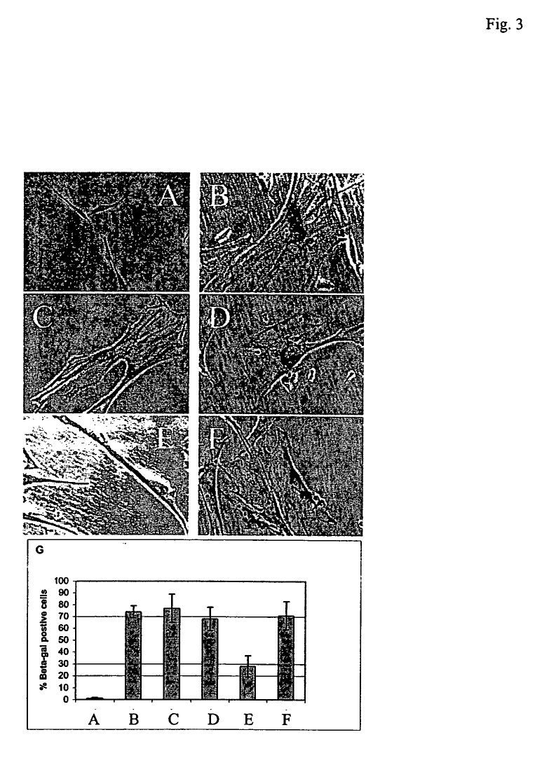

Cells were grown on films of denatured collagen. When PD30 and PD64 cells are grown on a matrix of 3 or 5 mg/ml denatured collagen, the morphology of aged cells is similar to that seen on non-coated culture dishes. This is shown in FIGS. 1C (PD30 cells (“A”) and PD64 cells (“B”) grown for six days on a film of 5 mg/ml (FIG. 1C) and 3 mg/ml (FIG. 1D) denatured collagen.

When aged cells (population doubling 64) are plated on dishes coated with lower concentrations of denatured collagen, the effect is quite striking. As shown in FIG. 1E, in which 0.5 mg/ml denatured collagen was used, by day six of culturing, cells on collagen appear much better organized morphologically and are significantly smaller than control cells. The effect becomes apparent with 1 mg/ml collagen and increases when collagen at lower concentrations (e.g., as low as 0.3 mg/ml) is used. The effect was much less apparent or not seen at all when native collagen at 0.5 mg/ml was used in similar experiments (FIG. 1F). The addition of various amounts of denatured collagen dissolved in PBS to tissue culture medium produced no effect. It appears, therefore, that growth of aged IMR90 cells on a matrix of denatured collagen of certain concentrations can result in the cells' rejuvenation.

Using morphological analysis, the effect of the denatured collagen matrix on the rate of aging in young cells was also assessed. To this end, passage 8 (approximately 24 population doublings) primary human fibroblasts IMR90 were maintained for 10 passages (approximately 30 population doublings) on denatured collagen film of 0.5 mg/ml and compared with the same cells maintained in non-coated tissue culture dishes. As can be seen in FIG. 2, passage 18 cells grown on collagen matrix (FIG. 2C) appear significantly younger morphologically than their counterparts that were maintained in non-coated tissue culture dishes (FIG. 2B). When the same cells were maintained for 10 passages on native collagen film of 0.5 mg/ml, practically no effect was seen, and the morphology of cells was similar to that seen on non-coated culture dishes (FIG. 2D). These results indicate that growth on denatured collagen matrix can reduce the rate of cell aging.

### EXAMPLE 10

### β-galactosidase Expression is Ceased or Decreased in Aged Cells and Delayed or Prevented in Young Cells Growth on a Denatured Collagen Matrix

To further study the phenomenon of apparent rejuvenation by growth on a denatured collagen matrix, changes in several well-defined molecular markers of aging were assessed. The first marker analyzed was β-galactosidase. A previous study (Dimri et al., 1995) demonstrated that several types of human cells, upon approaching senescence at sub-confluent density, express a β-galactosidase (“SA”, for “senescence-associated” β-galactosidase) that is histochemically detectable at pH 6, and local blue precipitates are formed upon treatment with X-gal). It has also been shown that SA β-galactosidase staining in confluent young fibroblasts disappears 24-48 hours after replating at sub-confluent density (Dimri et al., 1995).

Accordingly, aged cells were tested for a decrease in SA β-galactosidase staining in cultures grown on denatured collagen. PD64 cells were plated either on non-coated dishes or on dishes coated with 0.5 mg/ml of denatured collagen. Initial seeding conditions for both collagen coated and non-coated plates were optimized to assure non-confluency after 6 days of cultivation. Cells were tested six days later for the occurrence of SA β-galactosidase. As shown in FIG. 3, the majority of control cells (74%, the mean of three independent determinations, 1,000 cells per determination) were stained. In contrast, only the minority of cells grown on collagen (28%, the mean of triplicate determinations) were β-galactosidase positive. Moreover, among stained cells the intensity of staining was significantly lower than that seen in control cells. When cells were grown on denatured collagen films of 5 or 3 mg/ml, or on native collagen films of 0.5 mg/ml, little if any effect was seen (FIG. 3). Thus, the results with SA β-galactosidase are consistent with the notion that growth on a denatured collagen matrix of certain concentrations leads to rejuvenation of aged IMR90 cells.

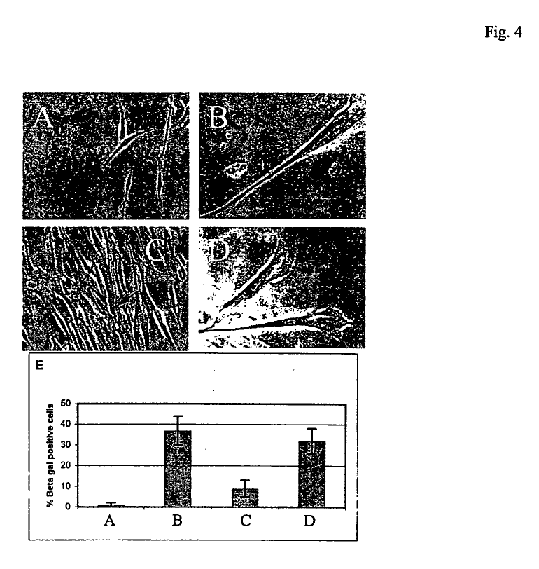

The expression of SA β-galactosidase was compared for cells grown for 10 passages (from PD24 to PD54) either on non-coated tissue culture dishes or on denatured collagen film of 0.5 mg/ml collagen. As shown in FIG. 4, in non-coated dishes SA β-galactosidase was seen in a sizable (37%, the mean of triplicate determinations) fraction of cells. In contrast, only minor (9%, the mean of triplicate determinations) fraction of cells grown on collagen matrix were stained for SA β-galactosidase. Moreover, when the same cells were maintained on native collagen matrix of 0.5 mg/ml, little, if any, effect was seen (FIG. 4). These results indicate that growth on denatured collagen matrix can significantly reduce the rate of cell aging.

### EXAMPLE 11

### Growth of Aged Cells on a Denatured Collagen Matrix Results in the Reduction of Oxidation in a Subset of Cellular Proteins

Oxygen-derived free radicals, generated by either environmental factors or during normal cellular metabolism, play an important role in cellular aging (Stadtman, 1992). Proteins are one of the major targets of oxygen free radicals and other reactive species. Oxidation modifies the side chains of methionine, histidine, and tyrosine and forms cysteine disulfide bonds (Stadtman, 1993). Metal-catalyzed oxidation of proteins introduces carbonyl groups at lysine, arginine, proline and threonine residues in a site-specific manner (Stadtman, 1993). The extent of protein oxidation was shown to reflect the degree of aging (Oliver et al., 1987; Starke-Reed and Oliver, 1989).

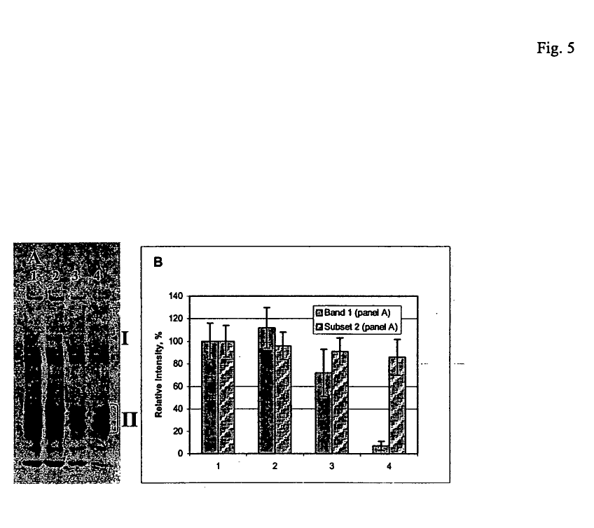

The extent of protein oxidation was assessed by testing for the presence of carbonyl groups to determine if in rejuvenated cells the pre-existent oxidized proteins are likely to be removed via proteosome action, which is a fairly fast process, and if matrix-mediated reduction in the extent of protein oxidation indeed takes place, it should be possible to observe. The carbonyl groups in the protein side chains were derivatized to 2,4-dinitrophenylhydrazone (DNP-hydrazone) by reaction with 2,4-dinitrophenylhydrazine. The DNP-derivatized protein samples were separated by polyacrylamide gel electrophoresis followed by Western transfer and immunoblotting with antibody specific to the DNP moiety of the proteins. The results of such an analysis are shown on FIG. 5. It is clear that growth of aged IMR90 cells on a denatured collagen matrix results in a significant reduction in the extent of oxidation of subsets of cellular proteins. As an example, a rather dramatic decrease in oxidation of one of the major protein bands was quantified (“I” in FIGS. 5A and 5B). Whereas the extent of oxidation of band “I” in aged cells grown on denatured collagen film of 0.5 mg/ml was only 7% (the mean of triplicate determinations in separate experiments) of that in control cells, the extent of oxidation of a subset of bands marked “II” in FIG. 5A decreased only slightly, to 86% (FIG. 5B). There are two possible reasons why this effect is seen primarily with subsets of total cellular proteins. First, this can reflect the specificity of oxidation (or rather of its reversal); second, certain pre-existent oxidized proteins can be removed from cells comparatively faster than other proteins. Consistent with other observations, little or no effect was seen when denatured collagen of high concentration or native collagen were used (FIG. 5). These results provide additional support to the notion that growth on a denatured collagen matrix of certain concentration leads to rejuvenation of aged IMR90 cells.

### EXAMPLE 12

### Growth of Aged Cells on a Denatured Collagen Matrix Results in the Restoration of the Expression of Hsp70 in Response to Stress

As stated above above, the ability to express a major stress response component, Hsp70, is attenuated in aged cells making them highly vulnerable when subjected to stresses. Importantly, the ability to express Hsp70 is not lost in aged cells but only suppressed, and can be restored (Volloch et al., 1998; Volloch and Rits, 1999).

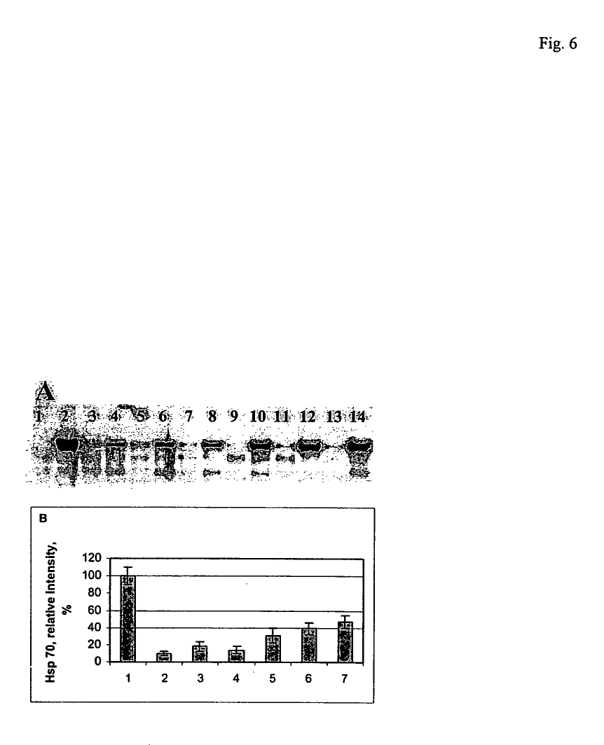

The possibility that cells are indeed rejuvenated by growth on denatured collagen matrix, and that the attenuation of Hsp70 expression in response to stresses can be reversed, was tested by growing aged IMR90 cells, PD64, either on regular dishes or on dishes coated with native collagen or denatured collagen at different concentrations. After six days of culturing, cells were subjected to a thermal stress (44° C., 30 minutes). Six hours later (to allow for potential accumulation of Hsp70 ) protein samples were prepared and subjected to SDS-polyacrylamide gel electrophoresis followed by Western transfer and immunoblotting with antibody (Spa810) specific for Hsp70. As shown in FIG. 6, very little Hsp70 is expressed in response to stress in control aged cells, whereas in cells grown on denatured collagen of high concentration (3 mg/ml) or in cells grown on native collagen of 0.5 mg/ml, the expression of Hsp70 is clearly induced in cells grown on denatured collagen of low concentrations. Indeed, in cells grown on denatured collagen matrix of 0.3 mg/ml the level of Hsp70 expression in response to stress reaches 47% (the mean of triplicate determinations in separate experiments) of that seen in young cells in response to the same stress (FIG. 6). As with the other effects described above, the expression of Hsp70 in aged cells in response to stresses was inversely proportional to concentrations of denatured collagen matrix, it increased with the decrease in concentration of denatured collagen and was only marginally affected by native collagen, it should be mentioned that in control experiments where various amounts of denatured collagen solution in PBS was added to the culture media, no similar effects were observed. Thus, the results with Hsp70 provide further show that growth on a denatured collagen matrix of certain concentration results in rejuvenation of aged IMR90 cells.

### EXAMPLE 13

### Maintenance of BMSCs on a Denatured Collagen Matrix Reduces the Rate of Cellular Aging-Related Morphological Changes

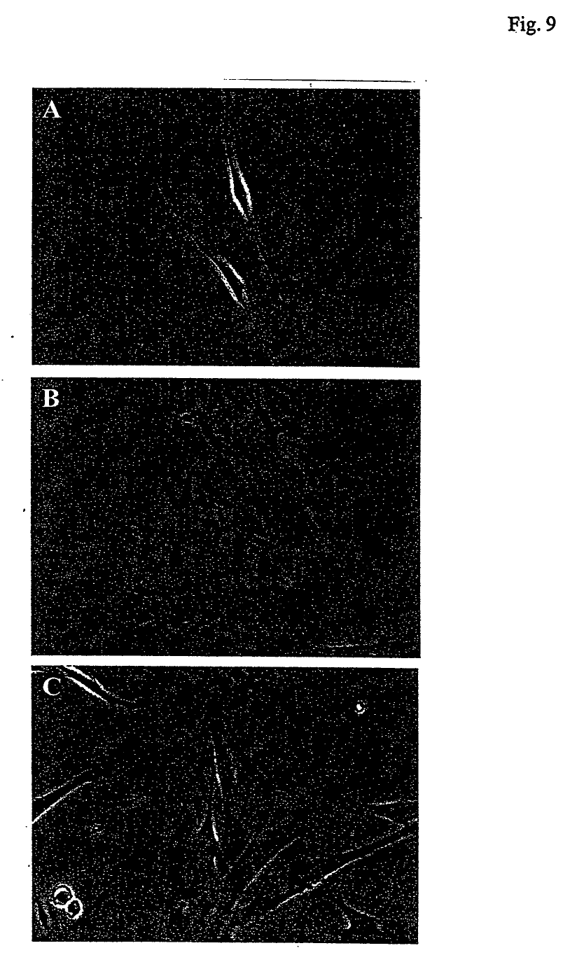

Cellular aging is defined by a number of cell divisions. With increasing number of population doublings (PDs) in culture, BMSCs undergo substantial morphological changes. Slim and morphologically well organized young cells gradually increase in size, spread on the dish and assume pancake-like appearance. “Young” BMSCs, as used in these Examples, are passage 2 cells, “Aged” cells are passage 11 cells cultivated on plastic, and/or passage 14 cells maintained on the denatured collagen I matrix. Passage 11 cells grown on tissue culture plastic occupied an area several times that occupied by young cells and were poorly defined morphologically. These results are shown in FIGS. 9A and 9B, which are a pair of photomicrographs. However, when maintained through passage 11 on a matrix of 0.5 mg/ml denatured collagen, the morphology of aged cells was strikingly different. As shown in FIG. 9C, which is a photomicrograph, cells on collagen appeared much better organized morphologically and significantly smaller than control aged cells. The effect is clearly matrix-dependent, as the addition of various amounts of denatured collagen dissolved in PBS to tissue culture medium produced no effect. This shows that growth of BMSCs on a matrix of denatured collagen results, at least at the morphological level, in the reduction of the rate of cellular aging.

### EXAMPLE 14

### Growth on a Denatured Collagen Matrix Results in the Retention of Inducibility of an Early Osteogenic Marker, Alkaline Phosphatase, in Response to Osteogenic Stimulants Treatment

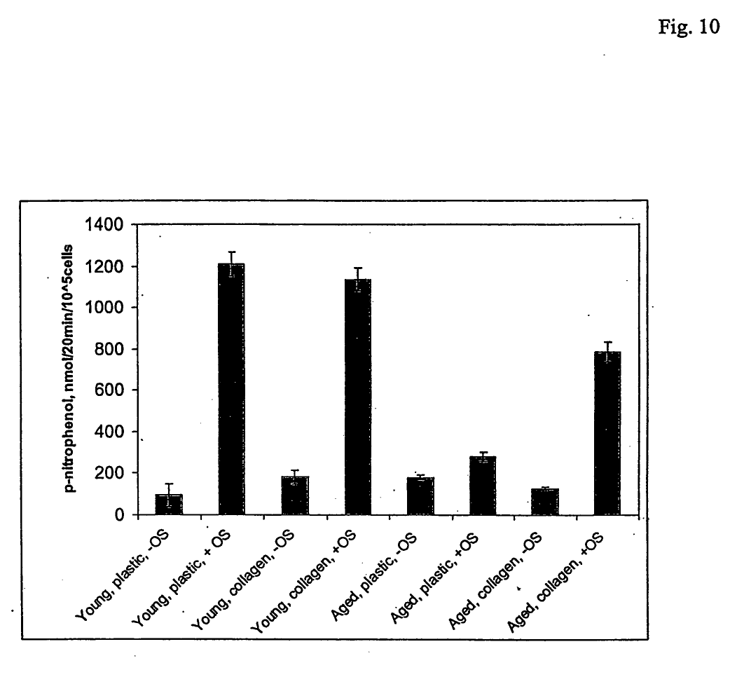

Alkaline phosphatase is one of the earliest markers expressed during osteogenic differentiation induced by ostegenic stimulants (OS). In human BMSCs it becomes detectable over the control at four days of treatment, peaks at 7-10 days, and recedes to control level past two weeks of treatment (Jaiswal et al., 1997). Levels of alkaline phosphatase were therefore measured at day 7 of OS treatment. The results are shown in FIG. 10, which is a graph. As can be seen in FIG. 10, young OS-treated cells exhibit similar levels of alkaline phosphatase when grown on plastic or maintained on the collagen matrix. It is also clear that the growth on the collagen matrix alone, without OS treatment, does not induce substantial alkaline phosphatase expression. On the other hand, in aged cells grown on plastic, the extent of alkaline phosphatase induction by OS treatment was greatly reduced, to 15% (mean of three independent determinations) of that seen in young OS-treated cells. In contrast, levels of alkaline phosphatase in OS-treated aged cells maintained on denatured collagen matrix were comparable (69%, mean of three independent determinations) with those seen in young OS-treated cells. Therefore, growth on a denatured collagen matrix preserves the potential for OS-mediated alkaline phosphatase expression in aged BMSCs.

### EXAMPLE 15

### Growth on a Denatured Collagen Matrix Preserves the Ability of BMSCs to Mineralize the Extracellular Matrix in Response to OS Treatment

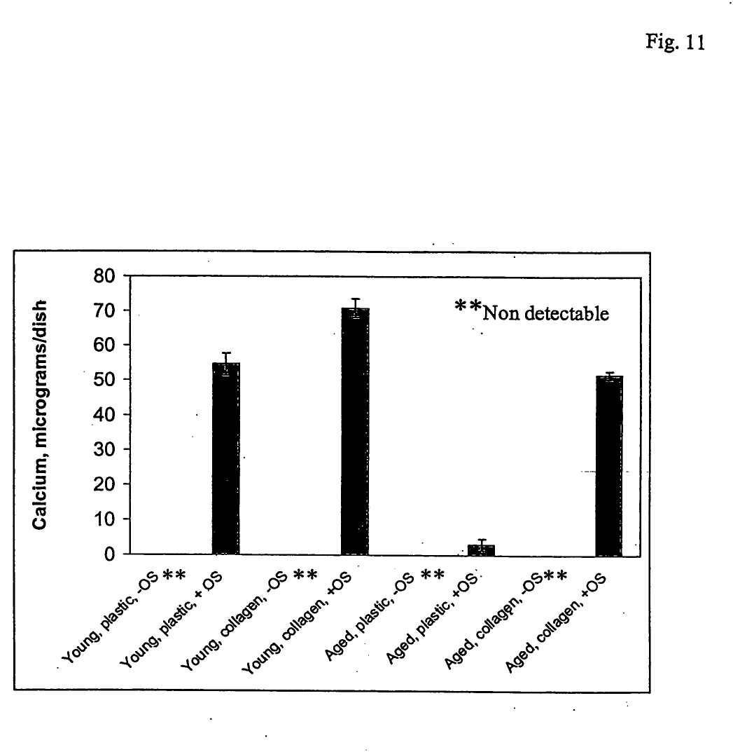

Young and aged BMSCs, grown either on plastic or cultivated on a denatured collagen matrix, were also analyzed for their potential to mineralize the extracellular matrix, which they exhibit when cultured in the presence of OS. The results are shown in FIG. 11, which is a graph. As shown in FIG. 11, at day 14 of OS treatment young cells grown on the collagen matrix deposited about one-third (29%, the mean of three independent determinations) more calcium than their counterparts grown on plastic. The effect is OS-dependent, and no detectable calcium was deposited by cells grown on collagen matrix in the absence of OS. The amount of calcium deposited by OS-treated aged cells grown on plastic was only a small fraction (5.5%, the mean of three independent determinations) of that seen with young OS-treated cells. In contrast, the amount of calcium deposited by OS-treated aged cells grown on denatured collagen matrix was comparable with that deposited by young OS-treated plastic-grown cells, and only slightly lower than the amount deposited by OS-treated young cells grown on the collagen matrix (73%, the mean of three independent determinations). Whereas control cultures remained flat throughout the experiments, by day 14 of OS treatment young treated cells formed multilayered structures typical for osteogenic differentiation in vitro (Jaiswal et al., 1997). Importantly, while OS-treated aged cultures grown on plastic remained flat, their counterparts grown on collagen formed multilayered structures by day 14, consistent with mineralization pattern. This shows that growth on a denatured collagen matrix preserves the ability of BMSCs to mineralize the extracellular matrix in response to OS treatment.

### EXAMPLE 16

### Growth on a Denatured Collagen Matrix Preserves the BMSC's Potential to Express “Late” Osteogenic-specific Gene, Bone Sialoprotein, in Response to OS Treatment

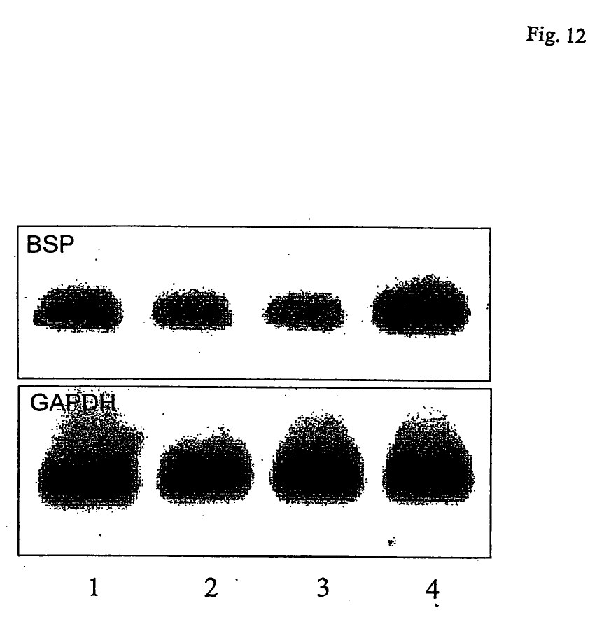

The ability of aged cells maintained either on plastic or on collagen matrices to express bone sialoprotein in response to OS treatment was assessed using RT-PCR technique. Bone sialoprotein (BSP) is a late osteogenic-specific marker (Chen et al., 1994; Aubin et al., 1995). The results are shown in FIG. 12, which is a graph. As shown in FIG. 12, OS treatment (14 days) induced very little, if any, expression of bone sialoprotein in aged cells grown on plastic. In contrast, a substantial increase in the levels of BSP-specific transcripts was seen in OS-treated cells maintained on collagen matrices. Thus, maintenance on the denatured collagen matrix preserves the BMSC's potential to express late osteogenic-specific genes in response to OS treatment.

### EXAMPLE 17

### Cultivation of Adult Human Bone Marrow Stromal Stem Cells (BMSCs) Preserves their Ability to Express the Major Stress-Protective Protein, Hsp70, in Response to Stress

Stress response is essential for cell viability, yet in human and animal cells it is attenuated in an age-dependent manner both in vivo and in vitro. In fact, the inability to express the major stress-protective protein Hsp70 in response to stresses is the primary cause of mortality of aged cells. Recently, it has been shown that BMSCs express signs of cellular aging during cultivation ex vivo. It was investigated whether such cultivation results in attenuation of Hsp70 expression in response to stress and whether growth on denatured collagen matrix prevents the loss of Hsp70 expression.

Early passage cells (thawed aliquot of passage one BMSC cells) and late passage cells (passage 8 cells started from a thawed aliquot of passage one cells) cultivated on plastic, and passage 11 of the same cells (cells grown on collagen exhibited much higher proliferative capacity and reached passage 11 at the same time that cells on plastic have undergone 8 passages) maintained on the denatured collagen matrix were either left untreated (control) or subjected to heat shock (44° C. for 45 min). Four hours later, cells were collected and analyzed by real time RT PCR for expression of Hsp70 mRNA, normalized for expression of housekeeping gene GAPDH).

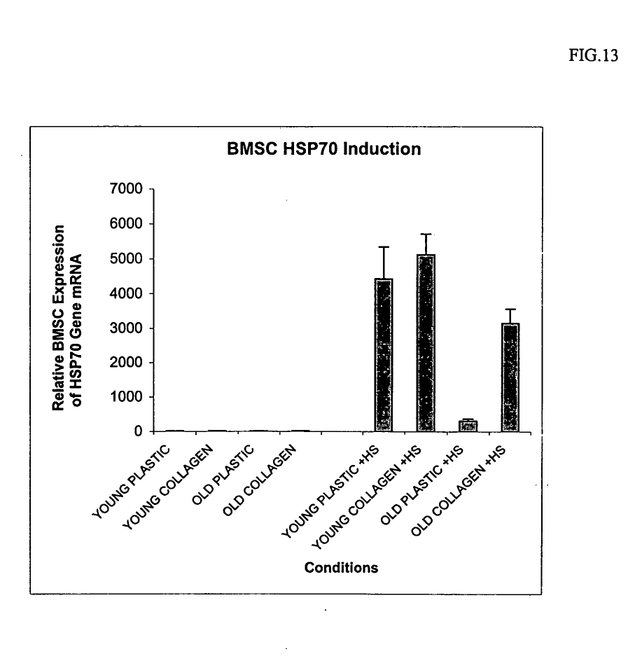

The results shown in FIG. 13 clearly indicate that during ex vivo expansion, growth on a denatured collagen matrix prevents loss of the ability to express Hsp70 in response to stress.

### EXAMPLE 18

### Serum Factor(s) are Essential but not Sufficient for Full Effect of Denatured Collagen Matrix on Differentiation Potential of Adult Human Bone Marrow Stromal Stem Cells (BMSCs)

To assess the input of serum into the observed effect of collagen-mediated retention of differentiation potential by BMSCs, the following experiment was performed. Early passage cells (thawed aliquot of passage one BMSC cells) and late passage cells (passage 8 cells started from a thawed aliquot of passage one cells) cultivated on plastic, and passage 11 of the same cells (cells grown on collagen exhibited much higher proliferative capacity and reached passage 11 at the same time that cells on plastic have undergone 8 passages) maintained on the denatured collagen matrix were induced by OS treatment toward osteogenic differentiation as described herein. During induction, late passage plastic-cultivated cells were maintained either on plastic or on denatured collagen matrix, and late passage collagen-cultivated cells were maintained on collagen matrix. In addition, from the beginning of OS treatment both treated and control cells were maintained either in regular medium (DMEM with 10% FCS) or in serum-free medium (DMEM supplemented with ITS+3).

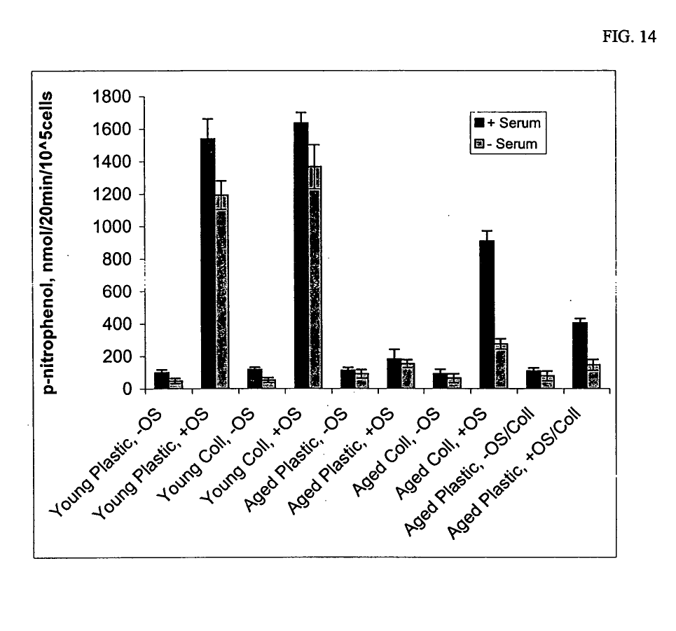

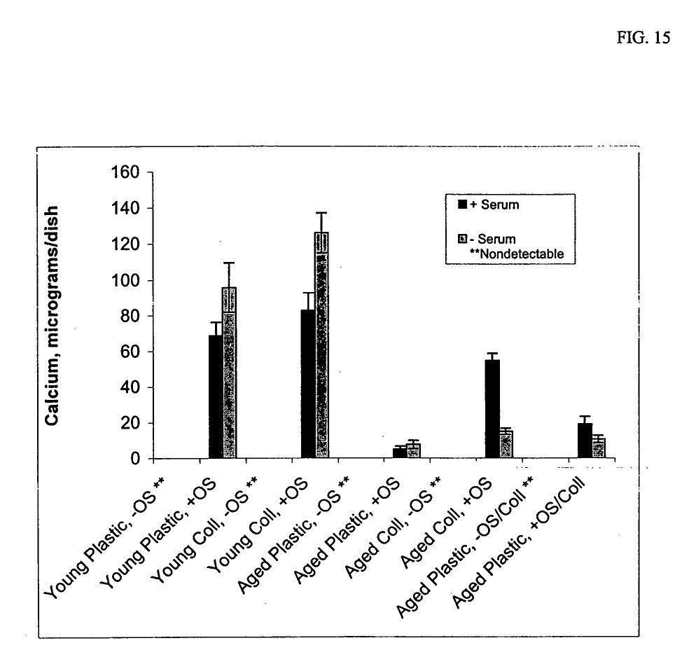

At day 10 of OS treatment, levels of alkaline phosphatase, one of the earliest markers expressed during OS-induced osteogenic differentiation, were measured. Alkaline phosphatase activity was calculated after measuring the absorbance of p-nitrophenol product, nmol/20 min/10⁵ cells, as described in Example 6. As can be seen in FIG. 14, early passage OS-treated cells maintained in the presence of serum exhibit comparable levels of alkaline phosphatase when either grown on plastic or maintained on the denatured collagen matrix. Similar results, albeit with lower levels of alkaline phosphatase, were seen with cells maintained in serum-free medium.

In late passage cells grown on plastic either in the presence or in the absence of serum, the extent of alkaline phosphatase induction by OS treatment was reduced to about 12% of that seen in early passage OS-treated cells. In contrast, levels of alkaline phosphatase in OS-treated late passage cells maintained on denatured collagen matrix in the presence of serum were at 56% of those seen in early passage OS-treated cells. However, OS-treated late passage cells maintained on denatured collagen matrix in the absence of serum retained only 19% of alkaline phosphatase levels seen in early passage OS-treated cells. Levels of alkaline phosphatase in OS-treated late passage plastic-cultivated cells maintained on denatured collagen matrix were at 44% in the presence of serum but only 21% in the absence of serum of those seen in OS/serum-treated late passage collagen-cultivated cells. The experiment thus demonstrates that (a) growth on denatured collagen matrix preserves the potential for OS-mediated alkaline phosphatase expression in ex vivo expanded BMSCs, and (b) the absence of serum significantly diminishes but does not eliminate the effect of collagen matrix.

At day 14 of OS teatment early and late passage BMSCs, grown on plastic or on a denatured collagen matrix either in the presence or in the absence of serum, were also analyzed for their ability to deposit extracellular calcium, in response to OS treatment, as an indicator of later stage osteogenic potential. As can be seen in FIG. 15, early passage OS-treated cells maintained in the presence of serum exhibit comparable levels of extracellular calcium when either grown on plastic or maintained on the denatured collagen matrix (in fact, collagen maintained cells deposited about 20% more calcium than their plastic maintained counterparts). A similar trend was seen in the absence of serum, although, surprisingly, in both cases cells deposited significantly more calcium in the absence than in then presence of serum. On the other hand, in late passage cells grown on plastic either in the presence or in the absence of serum, the extent of calcium deposition by induced OS treatment was reduced to about 7% of that seen in early passage OS-treated cells. In contrast, levels of extracellular calcium in OS-treated late passage cells maintained on denatured collagen matrix in the presence of serum were at 70% of those seen in early passage collagen-maintainedOS-treated cells. However, OS-treated late passage cells maintained on denatured collagen matrix in the absence of serum retained only 20% of levels of extracellular calcium seen in early passage OS-treated cells. Levels of extracellular calcium in OS-treated late passage plastic-cultivated cells maintained on denatured collagen matrix were at 36% in the presence on serum but only 19% in the absence of serum of those seen in OS/serum-treated late passage collagen-cultivated cells. The experiment demonstrates that (a) growth on denatured collagen matrix preserves the potential for OS-mediated extracellular calcium expression in ex vivo expanded BMSCs, and (b) the absence of serum significantly diminishes but does not eliminate the effect of collagen matrix. It seems that in addition to serum, another factor(s), such as for example, ECM differentially deposited on denatured collagen matrix versus plastic, may be required for the full effect of collagen matrices on retention of the differentiation potential. of BMSCs.

References:

Aoshiba, K., Rennard, S. and Spurzem, J. (1997) Cell matrix and cell-cell interaction modulate apoptosis of bronchial epitheleal cells. Am. J Physiol. 272:L28-37.

Aubin, J. E., Liu, F., Malaval, L., Gupta, A. K. (1995). Osteoblast and chondroblast differentiation. Bone 17(2 Suppl):77S-83S.

Bruder, S., Jaiswal, N., Haynesworth, S. (1997) Growth kinetics, self-renewal, and the osteogenic potential of purified human mesenchymal stem cellsduring extensive subcultivation and cryopreservation. J. Cell. Biochem. 64:278-294.

Cao, L., Lee, V., Adams, M., et al. (1999) Beta integrin-collagen interaction reduces chondrocyte apoptosis. Matrix Biol. 18:343-355.

Caplan, A., Syftestad, G., Osdoby, P. (1983) The development of embryonic bone and cartilage in tissue culture. Clin. Orthop. Re. Res. 174:243-263.

Chen J, McKee M D, Nanci A, Sodek J. (1994) Bone sialoprotein MRNA expression and ultrastructural localization in fetal porcine calvarial bone: comparisons with osteopontin. Histochem. J. 26(1):67-78.

Chen, C. S., Mrksich, M., Huang, S., Whitesides, G. M. and Ingber, D. E. (1997) Geometric control of cell life and death. Science 276:1425-1428.

Dimri, G., et al. (1995) A biomarker that identifies senescent human cells in culture and in aging skin in vivo. Proc. Natl. Acad. Sci. USA 92:9363-9367.

Galagher, J., Gundle, R., Beresford, J. (1996) Isolation and culture of human bone-derived cells. In “Methods in Molecular Biology”, Jones, G., ed. Humana Press.

Haynesworth, S., Baber, M., Caplan, A. (1992a) Cell surface antigens on human marrow-derived mesenchymal cells are detected by monoclonal antybodies. Bone 13:69-80.

Haynesworth, S., Goshima, J., Goldberg, V., Caplan, A. (1992b) Characterization of cells with osteogenic potential from human marrow. Bone 13:81-88.

Henriet, P., Zhong, Z., Brooks, P., Weinberg, K. and DeClerk, Y. (2000) Contact with fibrillar collagen inhibits melanoma cell proliferation by upregulating p27KIP1. Proc. NatI. Acad. Sci. USA 97:10026-10031.

Howell, S. and Doane, K. (1998) Collagen increases cell survival and prevents anti-beta 1 integrin-mediated apoptosis. Exp. Cell Res. 241:230-241.

Hoyt, D., Mannix, R., Rusnak, J., Pill, B. and Lazo, J. (1995) Collagen is a survival factor against LPS-induced apoptosisin cultured sheep pulmonary artery endothelial cells. Am. J. Physiol. 269:L171-177.

Jaiswal, N, Haynesworth, S., Caplan, A. Bruder, S. (1997) Osteogenic differentiation of purified, culture-expanded human mesenchymal cells in vitro. J. Cell. Biochem. 64:295-312.

LeBoy, P., Beresford, J., Devlin, C., Owen, M. (1991) Dexamethasone induction of osteoblast mRNA in rat marrow stromal cells culture. J. Cell. Physiol. 146:370-378.

Maniatopolous, C., Sodek., J., Melcher, A. (1988) Bone formation in vitro by stromal cells obtained from bone marrow of young adult rats. Cell Tissue Res. 254:317-330.

Mooney, A., Jackson, K., Bacon, R., et al. (1999) Collagen and laminin regulate glomerular mesangial cell susceptibility to apoptosis. Am. J. Pathol. 155:599-606.

Oliver, C., et al. (1987) Age related changes in oxidized proteins. J. Biol. Chem. 262:5488-5484.

Payne, K. and Veis, A. (1988) Fourier transform IR spectroscopy of collagen and gelatin solutions: deconvolution of the amide I band for conformational studies. Biopolymers 27:1749-60.

Ranucci, C. S. and Moghe, P. V. (2001) Substrate microtopography can enhance cell adhesive and migratory responsiveness to matrix ligand density. J. Biomed. Mater. Res. 54:149-161.

Rezania, A. and Healy, K. E. (2000) The effect of peptide surface density on mineralization of a matrix deposited by osteogenic cells. J. Biomed. Mater. Res. 52:595-600.

Stadtman, E. (1992) Protein oxidation and aging. Science 257:1220.

Stadtman, E. (1993) Oxidation of free aminoacids and amino acid residues in proteins. Ann. Rev. Biochem. 62:797-816.

Starke-Reed, P. and Oliver, C. (1989) Protein oxidation and proteolysis during aging and oxidative stress. Arch. Biochem. Biophys. 275:559-568.

Volloch ,V., Mosser, D., Massie, B. and Sherman, M. (1998) Reduced Thermotolerance in aged cells results from a loss of an Hsp70-mediated control of JNK signaling pathway. Cell Stress Chaperones. 3(4):265-71.

Volloch, V. and Rits, S. (1999) A natural extracellular factor that induces Hsp70, inhibits apoptosis, and restores stress resistance in aged human cells. Exp. Cell Res. 253(2):483-492.

Walker, G., Walker, D. and Lithgow, G. (1998) Genes that determine both Thermotolerance and rate of aging in C. elegans. Ann. NY Acad. Sci. 851:444-449.

All patents, patent applications, and published references cited herein are hereby incorporated by reference in their entirety. While this invention has been particularly shown and described with references to preferred embodiments thereof, it will be understood by those skilled in the art that various changes in form and details may be made therein without departing from the scope of the invention encompassed by the appended claims.

1. A method of preserving one or more cellular functions that are characteristic of cells in a non-senescent state, wherein the one or more cellular functions are lost in cells that are in a senescent state, the method comprising
(a) providing cells that possess one or more cellular functions that are characteristic of cells in a non-senescent state;
(b) providing a matrix of denatured biocompatible polymer; and
(c) culturing the cells of (a) on the matrix of (b) under conditions sufficient to preserve the one or more cellular functions that are characteristic of cells in a non-senescent state;
thereby preserving the one or more cellular functions that are characteristic of cells in a non-senescent state.

2. A method of restoring one or more cellular functions that are characteristic of cells in a non-senescent state, wherein the one or more cellular functions are lost in cells that are in a senescent state, the method comprising
(a) providing cells that have lost one or more cellular functions that are characteristic of cells in a non-senescent state;
(b) providing a matrix of denatured biocompatible polymer; and
(c) culturing the cells of (a) on the matrix of (b) under conditions sufficient to restore the one or more cellular functions that are characteristic of cells in a non-senescent state;
thereby restoring the one or more cellular functions that are characteristic of cells in a non-senescent state.

3-4. (canceled)

5. A method of preserving the plasticity of cells, the method comprising
(a) providing cells that possess plasticity;
(b) providing a matrix of denatured biocompatible polymer; and
(c) culturing the cells of (a) on the matrix of (b) under conditions sufficient to preserve the plasticity of the cells;
thereby preserving the plasticity of the cells.

6-8. (canceled)

9. The method of claim 1, wherein the cellular function is selected from the group consisting of: plasticity, differentiation potential, β-galactosidase expression, alkaline phosphatase expression, bone sialoprotein expression, calcium deposition, and heat shock protein expression.

10. (canceled)

11. The method of claim 1, wherein the denatured biocompatible polymer is selected from the group consisting of: fibrous proteins, polyesters and polysaccharides.

12. The method of claim 11, wherein the fibrous protein is selected from the group consisting of: collagen, silk, keratins, tubulins, actins, elastins and myosins.

13. The method of claim 11, wherein the polyester is selected from the group consisting of: polycaprolactone, polylactic acid, polyglycolic acid, polynucleic acids and polyhydroxyalkanoates.

14. The method of claim 11, wherein the polysaccharide is selected from the group consisting of: alginate, chitosan, chitin, gellan, pullulan, cellulose, hyaluronic acid, starch, amylose, amylopectin, pectin, glycogen, glycosaminoglycan, hyaluronate, chondroitin, heparin, dextrin, inulin, mannan, chitin.

15. (canceled)

16. The method of claim 12, wherein the collagen is type I collagen.

17. The method of claim 16, wherein the collagen is at a concentration of 0.1 mg/ml to 5 mg/ml.

18-19. (canceled)

20. A cell culture composition comprising a denatured biocompatible polymer, where the polymer is selected from the group consisting of: fibrous proteins, polyesters and polysaccharides.

21. The cell culture composition of claim 20, wherein the fibrous protein is selected from the group consisting of: collagen, silk, keratins, tubulins, actins, elastins and myosins.

22. The cell culture composition of claim 20, wherein the polyester is selected from the group consisting of: polycaprolactone, polylactic acid, polyglycolic acid, polynucleic acids and polyhydroxyalkanoates.

23. The cell culture composition of claim 20, wherein the polysaccharide is selected from the group consisting of: alginate, chitosan, chitin, gellan, pullulan, cellulose, hyaluronic acid, starch, amylose, amylopectin, pectin, glycogen, glycosaminoglycan, hyaluronate, chondroitin, heparin, dextrin, inulin, mannan, chitin.

24. The cell culture composition of claim 20, wherein the denatured biocompatible polymer is a mixture of polymers.

25. The cell culture composition of claim 21, wherein the collagen is type I collagen.

26. The cell culture composition of claim 25, wherein the collagen is denatured at 50° C. for 12 hours.

27. The cell culture composition of claim 26, wherein the composition is generated by evaporation of a collagen solution at a concentration of 0.1 mg/ml to 5 mg/ml.

28. The cell culture composition of claim 26, wherein the composition is generated by evaporation of a collagen solution at a concentration of 0.3 mg/ml.

29. The cell culture composition of claim 26, wherein the composition is generated by evaporation of a collagen solution at a concentration of 0.5 mg/ml.

30-32. (canceled)
